(19) United States
(12) Patent Application Publication
(10) Pub. No.: US 2023/0332136 A1
(43) Pub. Date: Oct. 19, 2023

(54) IL-17 RECEPTOR MODULATOR SCREENING SYSTEM

(71) Applicant: New York University, New York, NY (US)

(72) Inventors: Yuelin Song, New York, NY (US); Dan R. Littman, New York, NY (US)

(21) Appl. No.: 18/303,421

(22) Filed: Apr. 19, 2023

Related U.S. Application Data
(60) Provisional application No. 63/332,650, filed on Apr. 19, 2022.

(51) Int. Cl. C12N 15/10

(52) U.S. Cl. CPC C12N 15/1055

(57) ABSTRACT

Provided are compositions and methods for identifying agents that can modulate IL-17 receptors. The compositions and methods use modified cells that produce a detectable signal that indicates whether or not a test agent is an agonist or antagonist of and IL-17 receptor.

### CROSS REFERENCE TO RELATED APPLICATION

This application claims priority to U.S. provisional patent application No. 63/332,650, filed Apr. 19, 2022, the entire disclosure of which is incorporated herein by reference.

### FIELD

The disclosure relates generally to systems, compositions and method for identifying agents that can modulate Interleukin-17 (IL-17) receptors.

### BACKGROUND

IL-17 family cytokines are evolutionarily ancient and highly conserved across species. These unique cytokines play important roles in host defense as well as allergic reactions and autoimmune diseases. Among IL-17 family cytokines, IL-17A/F, IL-17E (IL-25), and IL-17C require intracellular adaptor protein Act1 to be recruited to their cognate receptor heteromer complexes (IL-17RA/RC, IL-17RA/RB, IL-17RA/RE, respectively) to signal to diverse downstream pathways in a cell-type dependent manner. Because of the pervasive role that IL-17 family cytokines play in autoimmunity and, potentially, in metabolic and neurological disease, there has been a significant amount of effort in developing IL-17 cytokine blockade for treating diseases such as rheumatoid arthritis, psoriasis, atopic dermatitis, inflammatory bowel disease, and others. So far, only antagonistic biological agents such as neutralizing antibodies are available, but no small molecule modulators have been successfully identified to specifically and effectively modulate IL-17 receptor signaling. Therefore, there is an ongoing and unmet need for new compositions and methods to identify agents that can modulate IL-17 receptors. The present disclosure is pertinent to this need.

### BRIEF SUMMARY

The present disclosure compositions and methods to identify agents that can modulate IL-17 receptors. The compositions include modified cells. The modified cells are modified to express an IL-17 receptor that forms a heteromer comprising separate proteins upon activation by ligand binding. At least one of the proteins that forms the heteromer is configured as a first fusion protein that includes a protease recognition site and a transcription transactivator segment. The protease recognition site is configured to liberate the transcription transactivator upon protease cleavage of the protease recognition site. The modified cells are also modified to express an activating protein configured as a second fusion protein. The second fusion protein localizes to the heteromer when formed. The second fusion protein comprises an activating protein and a protease that is functional on the protease recognition site. The modified cells are also modified to comprise a sequence encoding a reporter protein that is operably linked to a promoter that is functional with the transcriptional transactivator once liberated from the first fusion protein such that expression of the reporter protein driven by the liberated transcriptional activator signifies activation of the receptor. The modified cells can be used to detect modulation of an IL-7 receptors that can be activated by IL-17A, IL-17F, IL-17E, IL-17C, or a combination thereof.

In embodiments, the described first fusion protein comprises a transcription transactivator that is functional with a tetracycline responsive element. In embodiments, the second fusion protein comprises an Act1-tobacco etch virus (TEV) protease that is functional to cleave the first fusion protein to thereby liberate the transcription transactivator. In embodiments, the reporter protein participates in production of an optically detectable signal, which may be a fluorescent or chemiluminescent signal.

Methods provided by the disclosure comprise contacting the described modified cells with an agonist to produce expression of the reporter protein, and subsequently contacting the cells with one or more test agents. A decrease of a signal produced by the reporter protein that is caused by competition between a test agent and the agonist for binding to the IL-17 receptor signifies that the test agent is an antagonist of the IL-17 receptor. In another embodiment, the disclosure provides a method comprising contacting the described modified cells with one or more test agents. Detecting expression of the reporter protein signifies that the test agent is an agonist of the receptor.

### BRIEF DESCRIPTION OF THE FIGURES

The Figures of this disclosure provide illustrations of embodiments of the disclosure which are not meant to be limiting. The disclosure includes each component of each of the described modified proteins, each step of any process individually, and all combinations of steps.

The patent or application file contains at least one drawing executed in color. Copies of this patent or patent application publication with color drawing(s) will be provided by the Office upon request and payment of the necessary fee.

FIG. 1 provides a cartoon overview of Act1-dependent IL-17 receptor family signaling. IL-17RA is a shared receptor for different IL-17 family cytokine signaling. When paired with IL-17RC the IL17RA/RC heterodimer recognize IL-17A and IL-17F; when paired with IL-17RB, it recognizes IL-25; when paired with IL-17RE, it recognizes IL-17C. All downstream signaling is mediated by adaptor protein Act-1.

FIG. 2 provides a cartoon depicting embodiments of the disclosure where the IL-17 receptor complex is in an inactive state (left) and an active state (e.g., an activated receptor; (right)). Specifically in the ADIR-17 system (also applicable to other future ADIR systems) upon cognate ligand binding, Act-1 is recruited to interact with IL-17RC. The TEV protease fused with Act-1 will recognize the peptide sequence (TSC) that links IL-17RC and transactivator tTA and release tTA from IL-17RC. tTA will then translocate into the nucleus and binds to TRE promoter specifically driving the transcription of dmCherry.

FIGS. 3A and 3B provide construct schematics (designed for mouse genes), and cartoon depictions of the proteins encoded by the constructs, and a graph illustrating IL-17A titrations for activation of an endogenous target gene (IL-6) in MEF cells. The data show that the MEFs possess functional endogenous IL-17RA, which does not need to be provided exogenously to produce the reporter system. FIG. 3A: schematics of functional units of the retroviral transfer plasmid constructs used to build the mouse ADIR system. FIG. 3B: Data showing MEF cells harbor endogenous IL-17RC and dose-dependent respond to IL-17A treatment with upregulation of IL-6.

FIGS. 4A-4C provide time course cell flow cytometry data for the kinetics of reporter activation using destabilized-mCherry (dmCherry) as a reporter protein in an embodiment of the described method. FIG. 4A: mADIR MEF cells producing dmCherry signal 4 hr post treatment with indicated concentration of IL-17A (right) or vehicle (left). FIG. 4B: mADIR MEF cells producing dmCherry signal 21 hr post treatment with indicated concentration of IL-17A (right) or vehicle (left). FIG. 4C: mADIR MEF cells turning on dmCherry signal 27 hr post treatment with indicated concentration of IL-17A (right) or vehicle (left).

FIGS. 5A-5M provide time course cell flow cytometry data for dmCherry reporter protein following withdrawal of IL-17A, in an embodiment of the described method. FIG. 5A: mADIR MEF cells without IL-17 treatment 24 hr after plating. FIG. 5B: mADIR MEF cells with 50 ng/ml IL-17A treatment 24 hr after plating with 27.2% cells turning on dmCherry signal. FIG. 5C: mADIR MEF cells first treated with 50 ng/ml IL-17A for 24 hr, then IL-17A is withdrawn from the media for 1 hr. FIG. 5D: mADIR MEF cells first treated with 50 ng/ml IL-17A for 24 hr then IL-17A is withdrawn from the media for 2 hr. FIG. 5E: mADIR MEF cells first treated with 50 ng/ml IL-17A for 24 hr then IL-17A is withdrawn from the media for 3 hr. FIG. 5F: mADIR MEF cells first treated with 50 ng/ml IL-17A for 24 hr then IL-17A is withdrawn from the media for 4 hr. FIG. 5G: mADIR MEF cells first treated with 50 ng/ml IL-17A for 24 hr then IL-17A is withdrawn from the media for 5 hr. FIG. 5H: mADIR MEF cells first treated with 50 ng/ml IL-17A for 24 hr then IL-17A is withdrawn from the media for 6 hr. FIG. 5I: mADIR MEF cells first treated with 50 ng/ml IL-17A for 24 hr then IL-17A is withdrew from the media for 17 hr. FIG. 5J: mADIR MEF cells first treated with 50 ng/ml IL-17A for 24 hr then IL-17A is withdrawn from the media for 24 hr/1 day. FIG. 5K: mADIR MEF cells first treated with 50 ng/ml IL-17A for 24 hr then IL-17A is withdrawn from the media for 2 days. FIG. 5L: mADIR MEF cells first treated with 50 ng/ml IL-17A for 24 hr then IL-17A is withdrawn from the media for 3 days. FIG. 5M: quantifications and fitting of mCherry+ cell ratios in 5A-L data to a one-phase decay curve with Rsquare=0.987 using Prism. The results are based on ligand disassociation from receptors. The number of molecules that dissociate in a given time interval is proportional to the number that were bound at the beginning of that interval. Therefore, each individual molecule of ligand bound to a receptor has a certain probability of dissociating from the receptor in any small time interval. That probability does not increase as the ligand stays on the receptor longer. When radioactive isotopes decay, the number of atoms that decay in a given time interval is proportional to the number of undecayed atoms that were present at the beginning of the interval. Therefore, each individual atom has a certain probability of decaying in a given time interval, and that probability is constant. The probability that any particular atom will decay does not change over time. The total decay of the sample decreases with time because there are fewer and fewer non-decayed atoms. When drugs are metabolized by the liver or excreted by the kidney, the rate of metabolism or excretion is often proportional to the concentration of drug in the blood plasma. Each drug molecule has a certain probability of being metabolized or secreted in a small time interval. As the drug concentration goes down, the rate of its metabolism or excretion goes down as well.

FIGS. 6A-6J provide information on reporter sensitivity, with flow cytometry data at 24h, using dmCherry as a reporter protein in an embodiment of the described method and an IL-17A titration graph. FIG. 6A: mADIR-17 MEF cells treated with vehicle after 24 hrs. FIG. 6B: mADIR-17 MEF cells treated with 100/(2′7) ng/ml IL-17A after 24 hrs with 5.36% dmCherry signal produced. FIG. 6C: mADIR-17 MEF cells treated with 100/(2\hat{ }6) ng/ml IL-17A after 24 hrs with 8.69% dmCherry signal produced. FIG. 6D: mADIR-17 MEF cells treated with 100/(2\hat{ }5) ng/ml IL-17A after 24 hrs with 12.6% dmCherry signal produced. FIG. 6E: mADIR-17 MEF cells treated with 100/(2\hat{ }4) ng/ml IL-17A after 24 hrs with 17.6% dmCherry produced. FIG. 6F: mADIR-17 MEF cells treated with 100/(2\hat{ }3) ng/ml IL-17A after 24 hrs with 19.9% dmCherry signal produced. FIG. 6G: mADIR-17 MEF cells treated with 100/(2\hat{ }2) ng/ml IL-17A after 24 hrs with 21.9% dmCherry signal produced. FIG. 6H: mADIR-17 MEF cells treated with 100/2 ng/ml IL-17A after 24 hrs with 24.5% dmCherry signal produced. FIG. 6I: mADIR-17 MEF cells treated with 100 ng/ml IL-17A after 24 hrs with 25% dmCherry signal produced. FIG. 6J: quantifications and fitting of mCherry+ cell ratios in 6A-H data to a [Agonist] vs. response (three parameter) curve with Rsquare=0.9981 using Prism. (From Prism software documentation: many log(dose) vs. response curves follow standard symmetrical sigmoidal shape. The approach is designed to determine the EC50 of the agonist, e.g., the concentration that provokes a response half way between the basal (Bottom) response and the maximal (Top) response. This model assumes that the dose response curve has a standard slope, equal to a Hill slope (or slope factor) of 1.0. This is the slope expected when a ligand binds to a receptor following the law of mass action, and is the slope expected of a dose-response curve when the second messenger created by receptor stimulation binds to its receptor by the law of mass action. If there are insufficient data points the standard slope model is used, which may be referred to as a three parameter dose-response curve.

FIGS. 7A-7N provide information on reporter specificity, with flow cytometry data for dmCherry reporter protein activity in response to the indicated IL-17 ligands, showing that the IL-17RA/RC system is specific for IL-17A and IL-17F signaling with different affinities across multiple cytokine concentrations. FIG. 7A: mADIR-17 MEF cells treated with vehicle after 24 hrs. FIG. 7B: mADIR-17 MEF cells treated with 0.4 ng/ml IL-17A after 24 hrs with 5.75% dmCherry signal produced. FIG. 7C: mADIR-17 MEF cells treated with 2 ng/ml IL-17A after 24 hrs with 24.6% dmCherry signal produced. FIG. 7D: mADIR-17 MEF cells treated with 10 ng/ml IL-17A after 24 hrs with 43.6% dmCherry signal produced. FIG. 7E: mADIR-17 MEF cells treated with 50 ng/ml IL-17A after 24 hrs with 55.1% dmCherry signal produced. FIG. 7F: mADIR-17 MEF cells treated with 0.4 ng/ml IL-17F after 24 hrs with 0.48% dmCherry signal produced. FIG. 7G: mADIR-17 MEF cells treated with 2 ng/ml IL-17F after 24 hrs with 0.81% dmCherry signal produced. FIG. 7H: mADIR-17 MEF cells treated with 10 ng/ml IL-17F after 24 hrs with 3.32% dmCherry signal produced. FIG. 7I: mADIR-17 MEF cells treated with 50 ng/ml IL-17F after 24 hrs with 13.8% dmCherry signal produced. FIG. 7J-N: mADIR-17 MEF cells treated with any doses of IL-25 (IL17E, noncognate ligand) after 24 hrs with similar dmCherry background signal as vehicle-treated cells (FIG. 7A), showing no-responsiveness of mADIR MEF cells to noncognate ligand.

FIGS. 8A-8C provide data showing that the mechanism of reporter activation requires (1) engagement of an active cognate ligand and (2) a functional interaction between IL-17 receptor and Act-1 upon ligand binding. FIG. 8A: non-functional splice variant of IL-17RC lacking exon 8 (encoding sequences required to interact with cognate ligands) treated with vehicle (left) or with 50 ng/ml IL-17A after 24 hrs. FIG. 8A shows no determined difference in dmCherry signal between left and right showing the ADIR system requires functional interaction between IL-17RC and cognate ligands. FIG. 8B: truncation mutant of IL-17RC lacking the amino acids after 648, which exhibits impaired interaction with Act-1, treated vehicle (left) or 50 ng/ml IL-17A (right) after 24 hrs. FIG. 8B shows dmCherry signal response at a reduced degree compared to FIG. 8C, showing IL-17RC and Act-1 interaction is required for the ADIR system. FIG. 8C: Full length IL-17RC in the purified ADIR system (cells are have silenced by selecting out ADIR components) treated with vehicle (left) or 50 ng/ml IL-17A (right), with signal-to-noise ratio significantly higher than FIG. 8A, 8B, showing a fully functional ADIR system requires all shown components interacting.

FIG. 9 provides a graphical depiction of constructs for use with human IL-17 receptors. Similar retroviral transfer plasmid constructs as mouse, with human cDNA sequences replacing mouse cDNA. Exogenous hIL-17RA is provided for a functional hADIR system. hIL-17RC, RB, RE constructs are added last separately to MEF cells already stably transduced with the other three required components, generating 3 independent cell lines.

FIGS. 10A-10C provide a workflow diagram of high-throughput screening assays (for antagonists) using the described constructs. FIG. 10A: ADIR-17A/F cells in multi-well plates treated with cognate ligands, and an antagonist candidate library is applied by adding individual candidates into individual wells. After a time period, wells show inhibition of dmCherry signal only in ADIR-17A/F system contain specific antagonists; wells show inhibition of dmCherry signal in all ADIR systems contain false-positive antagonists (non-specific); wells treated with anti-IL-17A/F neutralizing antibodies serve as positive controls as dmCherry signal will be blocked in these wells. FIGS. 10B and 10C: Similar antagonist screening approach for AIDR-17E system and ADIR-17C system.

FIGS. 11A-11C provide a workflow diagram of high-throughput screening assays (for agonists in this Fig.) using the described constructs. FIG. 11A: An agonist candidate library is applied by adding individual candidates into individual wells of the ADIR-17A/F system. After a time period, wells show dmCherry signal only in ADIR-17A/F system contain specific agonists; wells show dmCherry signal in all ADIR systems contain false-positive agonists (non-specific); wells treated with IL-17A/F (cognate ligands) serve as positive controls as dmCherry signal will be produced in these wells. FIG. 11B-C: Similar agonist screening approach for AIDR-17E system and ADIR-17C system.

FIGS. 12A-12D demonstrate an activator screen using the hADIR-17 system. A selected hADIR-17 clone generates dose-dependent response to hIL-17A and hIL-17F with a dmCherry signal. Data also shows hADIR-17 does not respond to hIL-25, showing ligand specificity of the hADIR-17 system. FIG. 12A: From bottom to top, a clonal hADIR-17 cell line treated with vehicle or increasing doses of human IL-17A with indicated concentrations. Data showing this clone of hADIR-17 MEF cells respond with saturated signal-to-noise ratio with indicated concentrations. FIG. 12B: From bottom to top, a clonal hADIR-17 cell line treated with vehicle or increasing doses of human IL-17F with indicated concentrations. Data indicate that hIL-17F concentration the hADIR-17 cell responds within its dynamic range. FIG. 12C: From bottom to top, a clonal hADIR-17 cell line treated with vehicle or increasing doses of human IL-17A with indicated concentrations lower than FIG. 12A. Data showing this with indicated hIL-17A concentration the hADIR-17 cell line is responding within its dynamic range. FIG. 12D: Data showing the hADIR-17 system does not respond non-cognate ligand IL-25 treatment.

FIGS. 13A-13C provide negative controls for the hADIR-17 system showing that lack of any single component results in no response to hIL-17A/F. FIG. 13A: hADIR-17 system lacking hIL-17RC does not respond to hIL-17A/F treatment. FIG. 13B: hADIR-17 system lacking hAct-1 does not respond to hIL-17A/F treatment. FIG. 13C: hADIR-17 system lacking hIL-17RA does not respond to hIL-17A/F treatment.

FIGS. 14A-14C demonstrate an inhibitor screen using the hAIDR-17 system. hIL-17A neutralizing antibody efficiently blocks the dmCherry signal generated by hIL-17A in a dose-dependent manner, but has minimal effect on the dmCherry signal generated by hIL-17F in the hADIR-17 system (upper panels) or by hIL-25 in the hADIR-25 system (lower panel). FIG. 14A: hADIR-17 dmCherry signal induced by hIL-17A can be neutralized only by anti-IL-17A, but not other non-cognate neutralizing antibodies. FIG. 14B: hADIR-17 dmCherry signal induced by hIL-17A can be neutralized only by anti-IL-17A in a dose-dependent manner. FIG. 14C: Anti-IL-17A neutralizing antibody does not inhibit IL-25-induced dmCherry signal on hADIR-25 system, demonstrating that an inhibitor specific to IL-17A-IL-17RA/RC signaling can be screened out using these three representative rules: 1) Exhibiting superior neutralizing activity to cognate ligand/receptors; 2) Dose-depend neutralizing activity; 3) Does not inhibit non-cognate ligand/receptor interactions.

FIGS. 15A-15C 15 provide negative controls for the hADIR-25 system showing that lack of single components results in no response to hIL-25. FIG. 15A: hADIR-25 system does not respond to IL-25 when lacking hIL-17RB. FIG. 15B: hADIR-25 system does not respond to IL-25 when lacking hAct-1. FIG. 15C: hADIR-25 system does not respond to IL-25 when lacking hIL-17RA.

### DETAILED DESCRIPTION

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein and in the appended claims, the singular forms “a”, “and” and “the” include plural references unless the context clearly dictates otherwise.

The disclosure includes all polynucleotide sequences described herein, the DNA and RNA equivalents of all such polynucleotides, including but not limited to cDNA constructs, and all polypeptides encoded by the polynucleotides described herein. The disclosure includes all amino acid sequences described herein.

The sequence of any polynucleotide and amino acid sequence identified herein by way of a database entry reference is incorporated herein by reference as the sequence it exists in the database on the effective filing date of this application or patent.

Any one or combination of components and process steps described herein can be omitted from the claims. The disclosure includes all steps and reagents, and all combinations of steps reagents, described herein, and as depicted in the accompanying Figures. The described steps may be performed as described, including but not necessarily sequentially. Any described reagent(s) and step(s) may be excluded from the claims of this disclosure. As such, the described reagents, steps, and systems of this disclosure may comprise or consist of any one or combination of said reagents and steps. The disclosure also includes all periods of time, and all reaction conditions, all sample preparation methods, described herein.

Unless defined otherwise herein, all technical and scientific terms used in this disclosure have the same meaning as commonly understood by one of ordinary skill in the art to which this disclosure pertains. Where the term “ADIR” is used herein it means ACT1-Dependent IL-17 Receptor Modulator Screening System. The preface “m” means mouse and the prefix “h” means human.

The present disclosure includes variants of the described IL-17 receptor proteins and the described fusion proteins. Such variants may have an altered amino acid sequence relative to a reference sequence. In certain embodiments, the disclosure includes homologous proteins. Homology in certain embodiments comprises amino acid sequences that are 80%-99% similar in amino acid sequence across the entire length of the described proteins. In embodiments, homologous variants have at least 80%, 85%, 95%, or 99% identity relative to the described sequences across the entire length of a referenced sequence. In embodiments, IL-17 receptor proteins are homologous to human or mouse IL-17 receptor sequences. Vectors and polynucleotides for use in producing the modified cells are provided by the disclosure. The polynucleotides may comprise any sequence that selectively hybridizes to a polynucleotide encoding the described proteins. Cells comprising the expression vectors are included in the disclosure. The disclosure also includes the described methods of making the modified cells. The disclosure also includes expression vectors that can be used to generate the described modified cells.

The disclosure provides modified cells for use in a method for identifying modulators of IL-17 receptors. In embodiments, the modified cells are modified such that they express a first and second fusion protein that form a heteromer. A “heteromer” such as an IL-17 receptor heteromer is a complex that includes at least two different proteins, such as the first and second fusion proteins described herein, and wherein the heteromer forms a functional IL-17 receptor. A “functional” IL-17 receptor means an IL-17 receptor that participates in signal transduction when bound by its cognate IL-17 receptor ligand. The heteromer may be a heterodimer, non-limiting depictions of which are depicted in the accompanying Figs. Thus, in general, the modified cells are modified such that they express an IL-17 receptor that forms a heteromer upon activation by ligand binding.

At least one of the proteins that forms the heteromer is configured as a first fusion protein that includes a protease recognition site and a transcription transactivator segment. The protease recognition site is configured to be cleavable by a protease to liberate the transcription transactivator upon protease cleavage of the protease recognition site. Another of the proteins that forms the heteromer comprises a second fusion protein. The second fusion protein comprises an activating protein and a protease that is functional on the protease recognition site of the first fusion protein to liberate the transcription transactivator. The modified cells also comprise a sequence that encodes a reporter protein that is operably linked to a promoter that is functional with the transcriptional transactivator once liberated from the first fusion protein. The liberated transcriptional transactivator therefore drives expression of the reporter protein. The disclosure includes pluralities of modified cells that express the same or different IL-17 receptors that comprise the first and second fusion proteins.

The IL-17 receptor components expressed by the cell may be specific for certain IL-17 ligands. In embodiments, the IL-17 receptor can be activated by IL-17A, IL-17F, IL-17E, IL-17C, or certain combinations thereof. As is known in the art, IL-17C binds to a heteromer comprising IL-17RA and IL-17RE. IL-17A and F bind to a heteromer comprising IL-17RA and IL-17RC. IL-17E (also referred to as IL-25) binds to a heteromer comprising IL-17RA and IL-17RB. In embodiments, the heteromer is thus a heterodimer.

The disclosure includes use of the described modified cells in methods of screening test agents for the ability to agonize or antagonize the described IL-17 receptors. This approach generally comprises exposing the modified cells to one or more test agents and determining whether or not the test agent promotes expression of the reporter protein. The approach is amenable to high-throughput approaches. The approach is also suitable for use with modified cells that express more than one isoform of a receptor, or with mixtures of cells that separately express different isoforms of the described receptors. Such systems can be adapted for assaying different receptors by, for example, using different transcription transactivators that drive expression of different reporter proteins that are operably linked to different promoters, each of which can be affected by the different transcription transactivators.

The type of test agent is not particularly limited. The test agent may be a drug, such as a small drug molecule, an antibody or antibody derivative, a peptide, an IL-17 mimic, or any other candidate IL-17 modulator. The test agents may be components of a library, such as a compound library, or an antibody or antibody derivative library. The test agents may be randomized peptides or proteins. The test agents may be unknown at the time the assay is performed, and compounds that modulate a reporter protein signal can be identified subsequent to performing the assay based on, for example, their location in a particular assay chamber.

Any segments of the described fusion proteins may include additional features to improve expression and/or function of the fusion proteins. For example, the modified cells may be modified to include any suitable nuclear localization signal for the transcription transactivator. Segments of the fusion proteins may also comprise amino acid sequences that comprise linking amino acids. Suitable amino acid linkers may be mainly composed of relatively small, neutral amino acids, such as glycine, serine, and alanine, and can include multiple copies of a sequence enriched in glycine and/or serine. In specific and non-limiting embodiments, the linker comprises 3, 4, 5, 6, 7, 8, 9, 10, 11, 12, 13, 14, 15, 16, 17, 18, 19, or 20 amino acids. In one example, the linker may be a glycine-serine linker.

In embodiments, a described fusion protein may comprise an amino acid sequence that facilitates discontinuous translation so that, for example, a reporter protein or other protein or peptide, can be produced from a single mRNA (although translation of the described proteins from different mRNAs may also be performed). In non-limiting examples, and internal ribosome entry site (IRES) can be used for discontinuous translation. In alternative embodiments, a peptide linker comprising any self-cleaving signal can be used, non-limiting embodiments of which include T2A, P2A, E2A, and F2A sequences, all of which are known in the art. The modified cells may be also modified to encode and express other components which may or may not be components of a described fusion protein. In embodiments, the modified cells thus may also express a selectable marker, such as a marker that confers resistance to a cytostatic or cytotoxic agent, or is an auxotrophic marker.

The type of promoter that is used is also not particularly limited, provided that it is functional with the transcription transactivator. In embodiments, the promoter is an endogenous promoter. In embodiments, the promoter is part of a contiguous polynucleotide sequence that is integrated into a chromosome of the modified cells such that the promoter is operatively linked to the sequence encoding the reporter protein, which may also be part of the integrated polynucleotide sequence. In one embodiment, the promoter comprises a promoter that is part of a viral vector or other polynucleotide delivery construct. In one embodiment, the promoter comprises an LTR promoter from a retrovirus vector.

Approaches to making the described modified cells include but are not limited to use of any a CRISPR system, the CRISPR system comprising at least one Cas enzyme and at least one guide RNA that targets a location in the cells wherein the described modification(s) is made. Any suitable CRISPR system may be used, non-limiting examples of which include Type I, II, Ill, IV, and Type V CRISPR systems. Other site directed nucleases may also be used, as can transposon-based systems. Alternatively, TALEN or zinc finger nucleases may be used to produce the modified cells.

The protease and protease recognition signal are not particularly limited. The system is illustrated using Act1-tobacco etch virus (TEV) protease and its cognate protease cleavage signal, but other proteases and protease recognition sites may be substituted, provided the protease can be configured so that it can perform its intended function on a suitable protease recognition site of the first fusion protein.

The type of reporter protein that the modified cells are engineered to express is not particularly limited. In certain examples, the reporter protein participates in producing a detectable signal. The detectable signal may be optically detectable. The detectable a signal may comprises UV light (<380 nm), visible light (380-740 nm), or far red light (>740 nm). In embodiments, the detectable signal comprises a fluorescent signal. In embodiments, the detectable signal is produced by a fluorescent protein, non-limiting examples of which include green fluorescent protein (GFP), enhanced GFP (eGFP), mCherry, blue fluorescent protein (BFP), and mVenus. In embodiments, the signal comprises a chemiluminescent signal, such as signals produced by luciferase or secreted alkaline phosphatase by acting on their cognate substrates.

The signal may be interpreted using any suitable device. In embodiments, any suitable imager located proximal to an analyzed sample can be used. In embodiments, free-space optics may be used to detect a signal from the described assay using any suitable signal detection device that is placed in proximity to the location where a detectable signal is generated, such as a CCD camera. In embodiments, the disclosure provides a device for use in sample analysis. In embodiments, the device may comprise, among other features, an optical waveguide to transmit a signal to any suitable measuring device such that optical accessibility to sample is not necessarily required to detect the signal. In embodiments, lens-less optics, and/or a cell phone based imaging approach is used. In embodiments, signal analysis is performed using a device that can be connected to or in communication with a digital processor and/or a computer running software to interpret the presence, absence, and/or intensity of a signal. The processor may run software and/or implement an algorithm to interpret an optically detectable signal, and generate a machine and/or user readable output. In an embodiment, an assay device used to perform the described analysis can be integrated or otherwise inserted into an adapter that comprises a detection device, such as a camera, or a microscope, including but not limited to a fluorescent microscope. In embodiments, a computer readable storage medium can be a component of an assay device of this disclosure, and can be used during or subsequent to performing any assay or one or more steps of any assay described herein. In embodiments the computer storage medium is a non-transitory medium, and thus can exclude signals, carrier waves, and other transitory signals.

In certain embodiments a result based on a determination of the presence, absence, intensity, or a combination thereof for the described is fixed in a tangible medium of expression, such as a digital file, and/or is saved on a portable memory device, or on a hard drive, or is communicated to a web-based or cloud-based storage system. The determination of the signal can be communicated a user for indexing, archiving, and identification of test agents that modulate or do not modulate the described receptors.

The method allows for analysis of a change in a variety of properties of the receptor that may arise due to the influence of the test agent on the particular type of receptor being analyzed. Such properties include but are not necessarily limited to receptor complex formation and receptor activation, such as signal transduction.

Determining a change in a property of any analyzed receptor that occurs in the presence of a test agent can include comparison to a control value, which may be obtained by any suitable approach. In embodiments, the control value comprises a property of the receptor in the absence of the test agent, or in the presence of a control agent with a known function on the receptor. In embodiments, the disclosure includes a control that is designed to identify general inhibitors of transcription, rather than specific receptor antagonists.

In embodiments, the disclosure pertains to testing a plurality of modified cell samples. The plurality of modified cell samples can be configured so as to be amenable for high throughput screening (HTS). In certain embodiments, the samples are divided into a plurality of reaction chambers, such as wells in a plate. Any multi-well plate or other suitable container can be used. In certain approaches, one or more 384-wells plates are used. In embodiments, detection of signals can be automated, such as by being analyzed by a robotized mechanism.

In embodiments, a multiplexed assay is used. The multiplexed assay can be performed using the same or differently modified cells in separate assay chambers. Alternatively, single cells may be modified for analyzing the effect of test agents on different receptors. In this approach, the modified cells may be modified as described herein, but wherein at least two different receptors are produced by the cells. The different receptors may be modified to, for example, use different transactivator proteins that drive expression of a second detectable protein which produces a signal that is different from the signal produced by a first detectable protein. Using this configuration, a test agent that modulates the effect of a first receptor can be differentiated from its effect, or the effect of another test agent, on the second receptor in the same cell. For instance, in a non-limiting embodiment, red and green fluorescent proteins can be used and will produce, upon receptor activation, a single green fluorescent signal, a single red fluorescent signal, or an overlapping red and green signal.

The same approaches apply to competition assays. In this approach, the method comprises first contacting the modified cells of with an agonist of the IL-17 receptor to produce expression of the reporter protein, and subsequently contacting the cells with one or more test agents. A decrease of a signal produced by the reporter protein that is caused by competition between the test agent and the agonist for binding to the IL-17 receptor signifies that the test agent is an antagonist of the IL-17 receptor. Thus, the system is readily adaptable for identifying IL-17 antagonists.

The disclosure also provides kit for performing the described methods. In embodiments, the kits comprise one or more sealed containers that comprise modified cells as described herein. The kits may also provide instructions on printed material that described how to use the modified cells to identify agents that are agonists or antagonists of IL-17 receptors.

The following Examples are intended to illustrate but not limit the disclosure.

### EXAMPLES

Embodiments of the disclosure are illustrated by the accompanying Figures.

Recombinant DNA plasmid constructs used in the examples used the following sequences as identified by GenBank accession numbers, the sequences from which are incorporated herein as they exist in the database as of the filing date of this application:
For mouse: IL-17RC: mRNA—NM_178942.1; Protein—NP_849273.1; Act-1 mRNA: NM_134000.3; protein—NP_598761.2;
For human: IL-17-cDNA: BC011624.2; Protein: AAH11624.1; IL-17RB cDNA: BC000980.1; Protein: AAH00980.1; IL-17RC; cDNA: AY359098.1; Protein: AAQ89456.1; Act-1 mRNA: NM_147200.3; Protein: NP_671733.2.

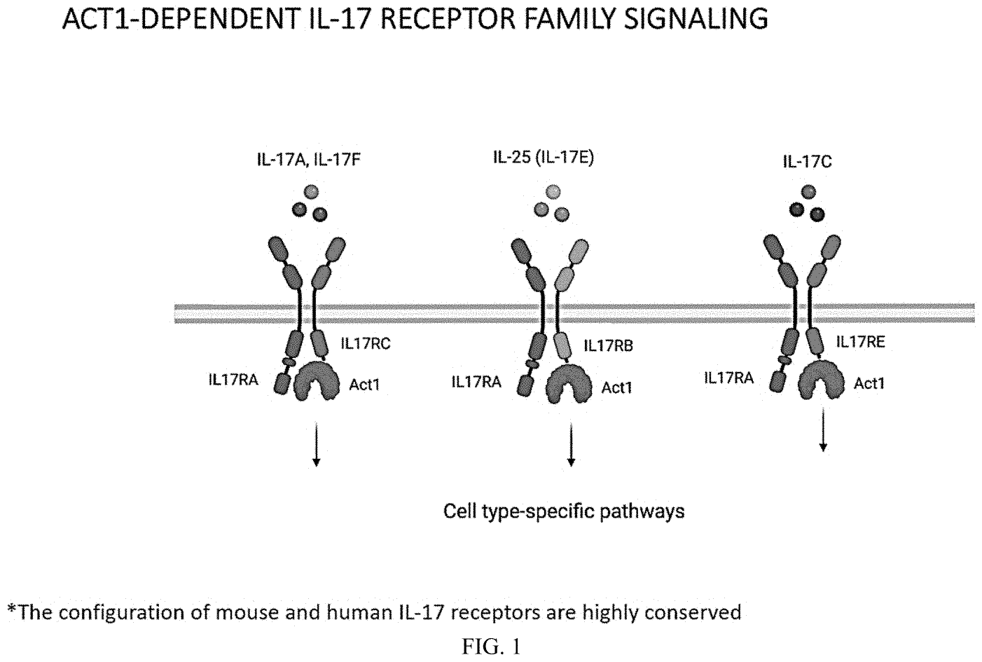

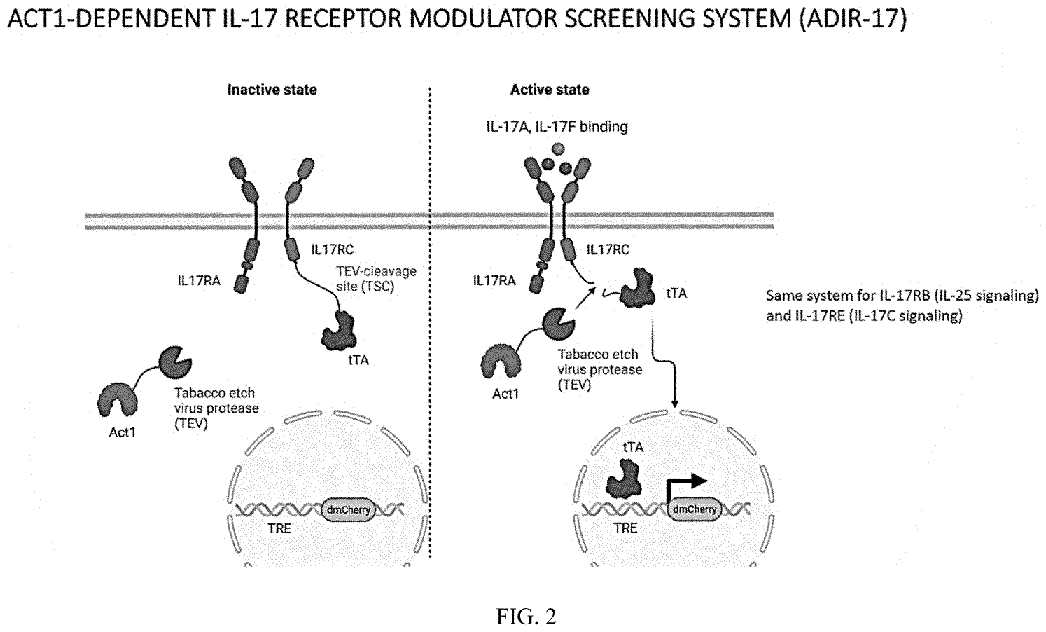

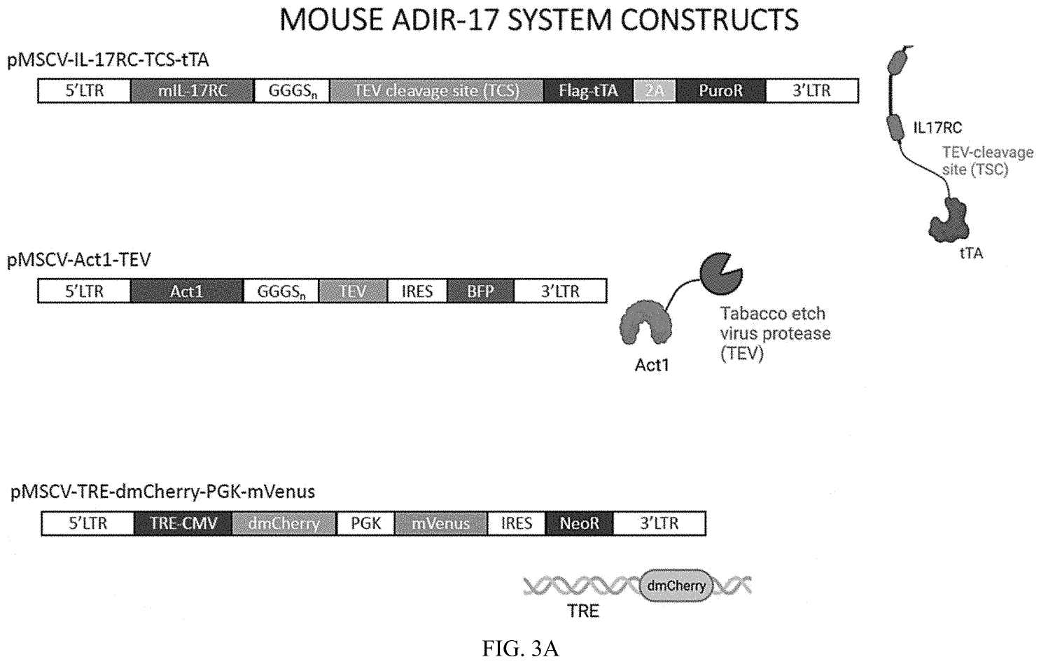

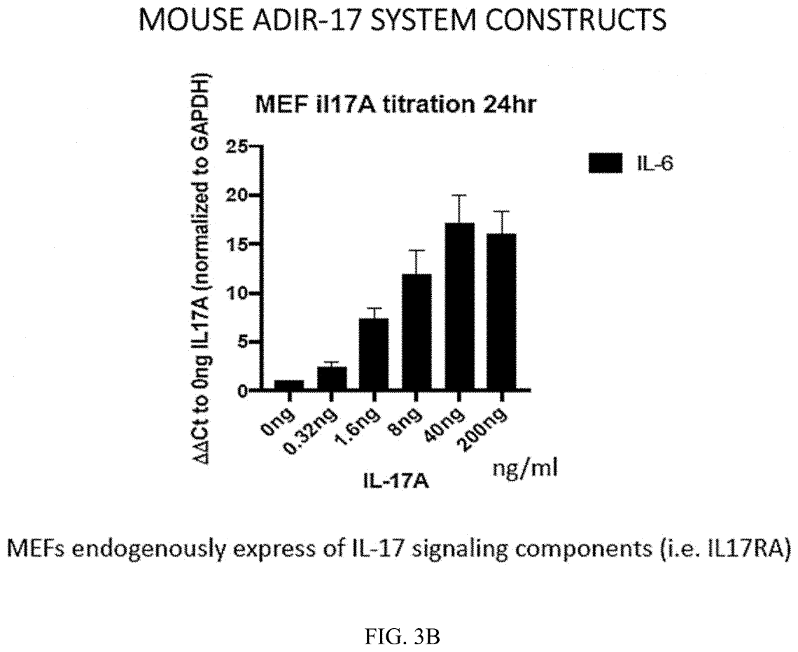

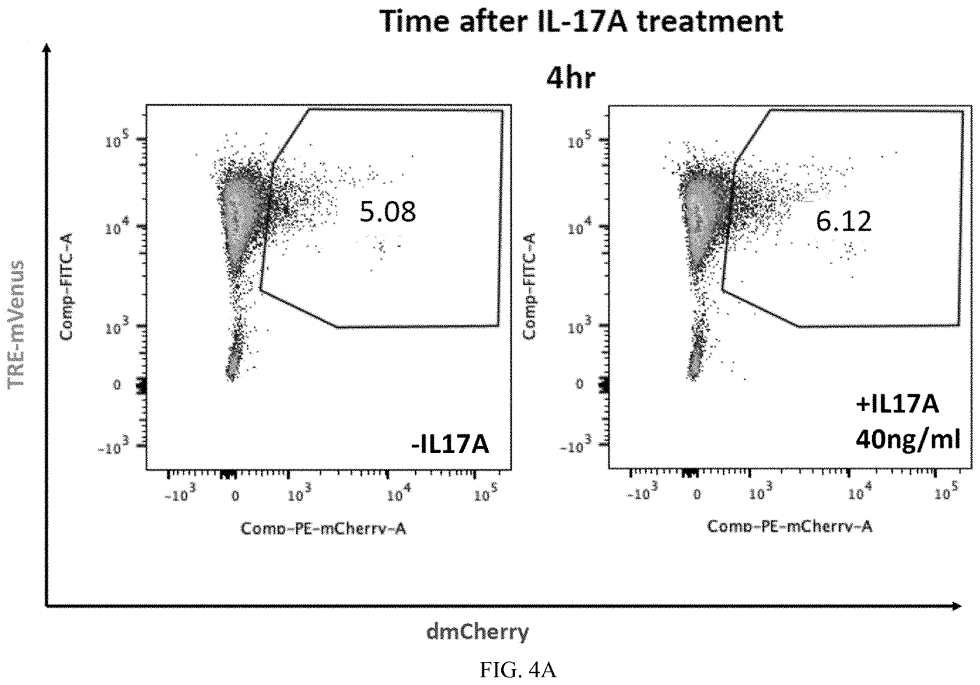

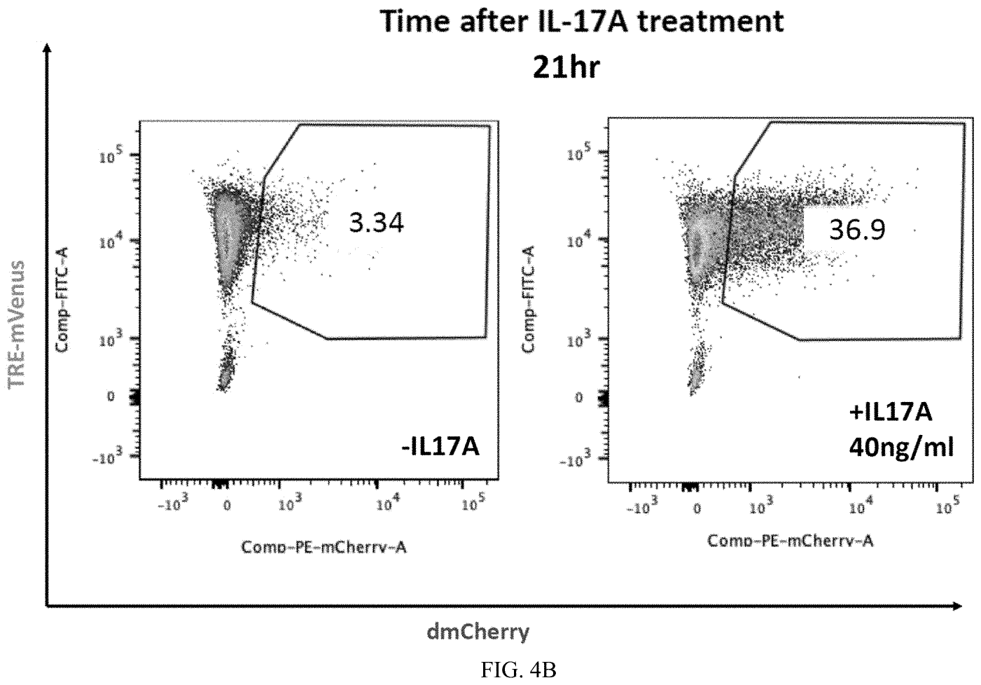

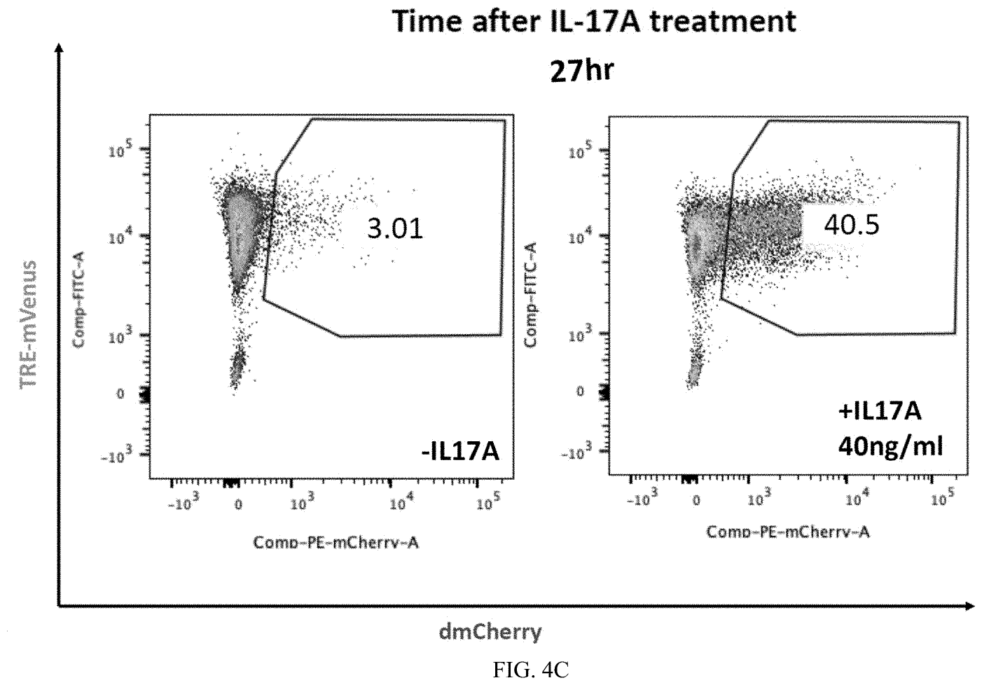

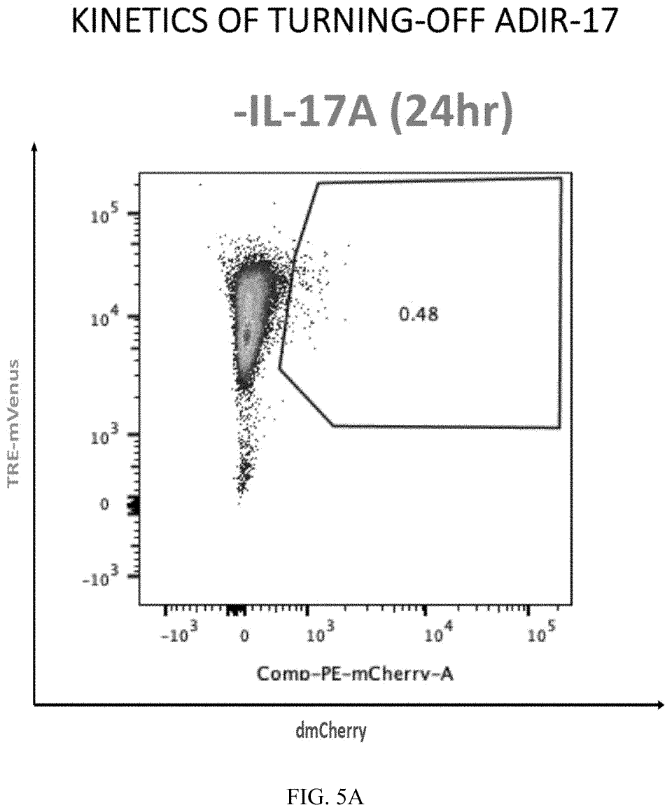

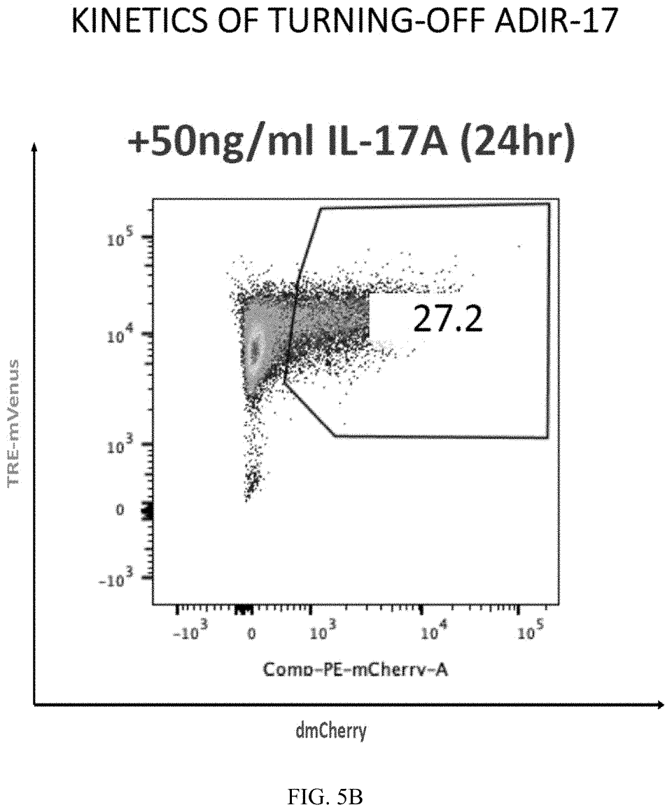

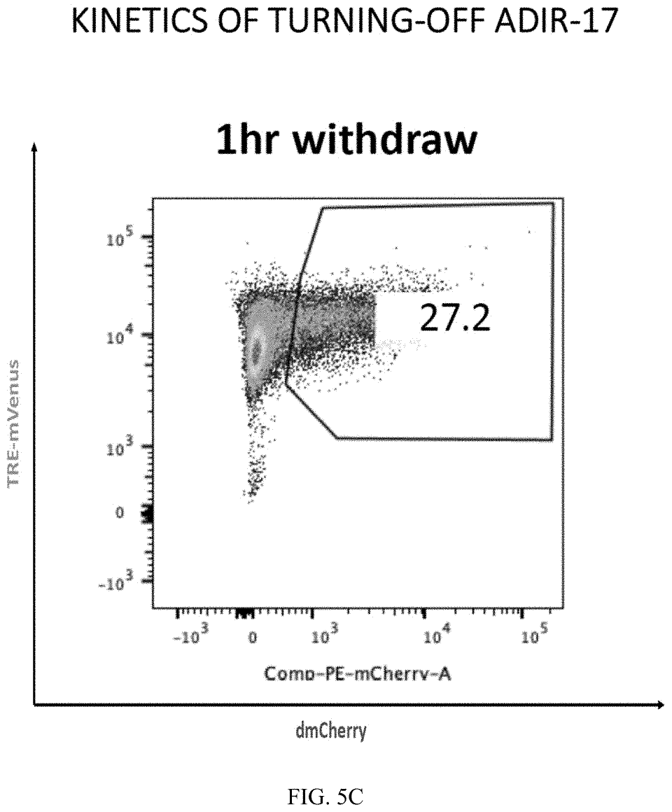

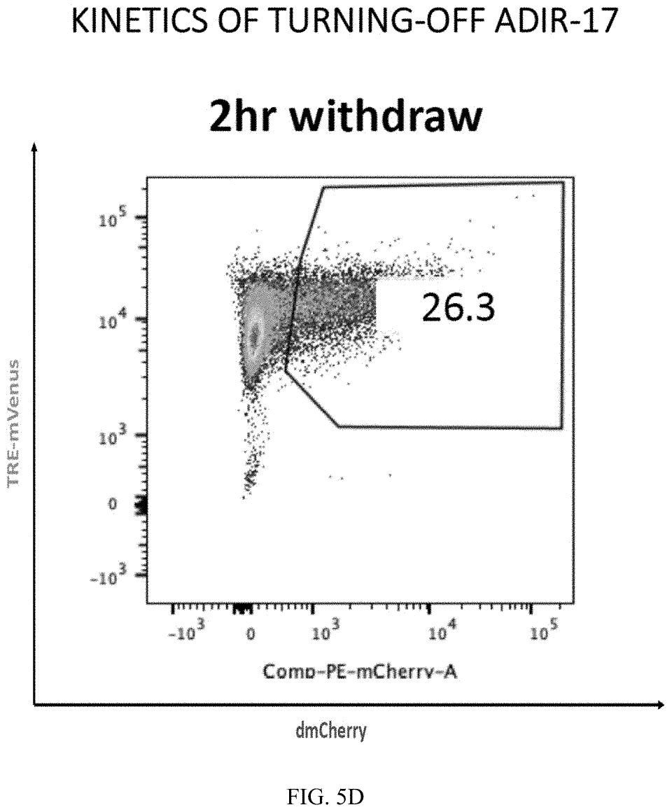

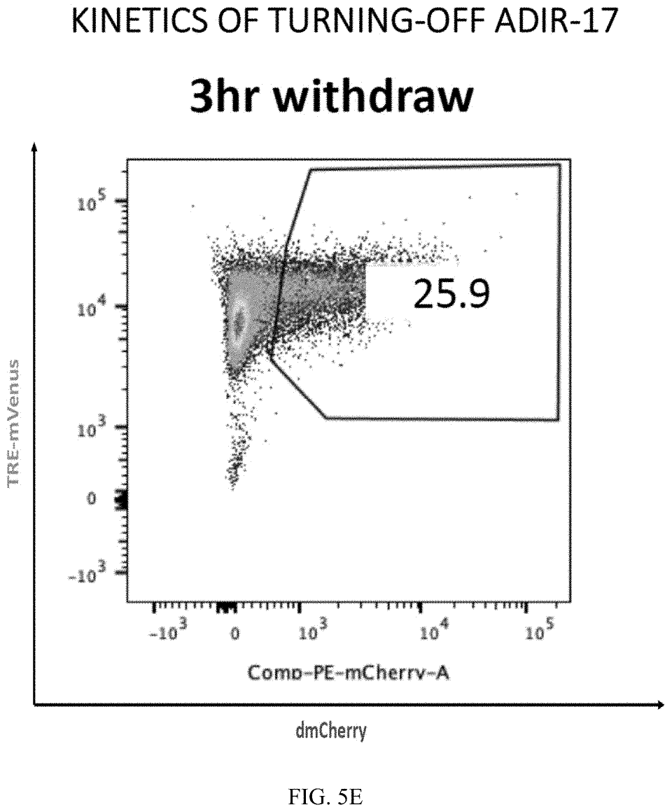

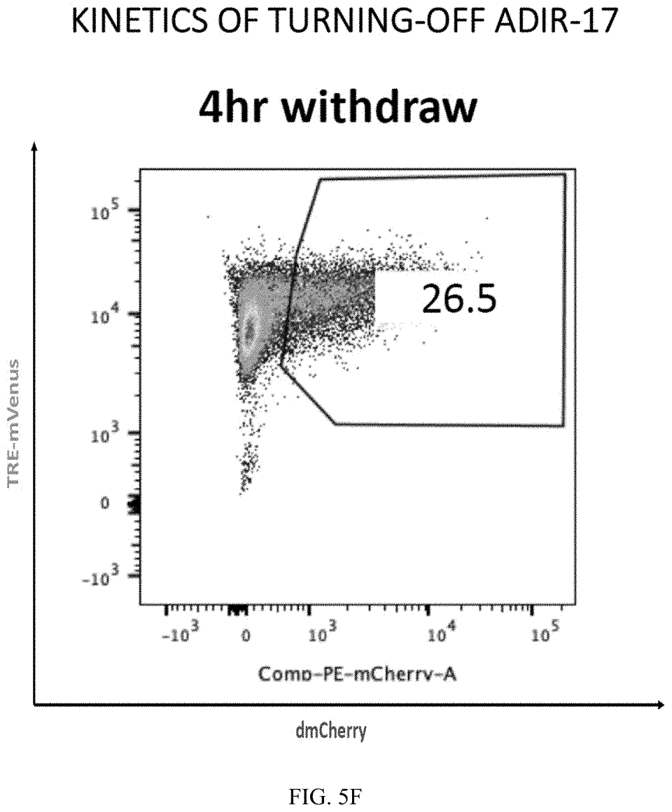

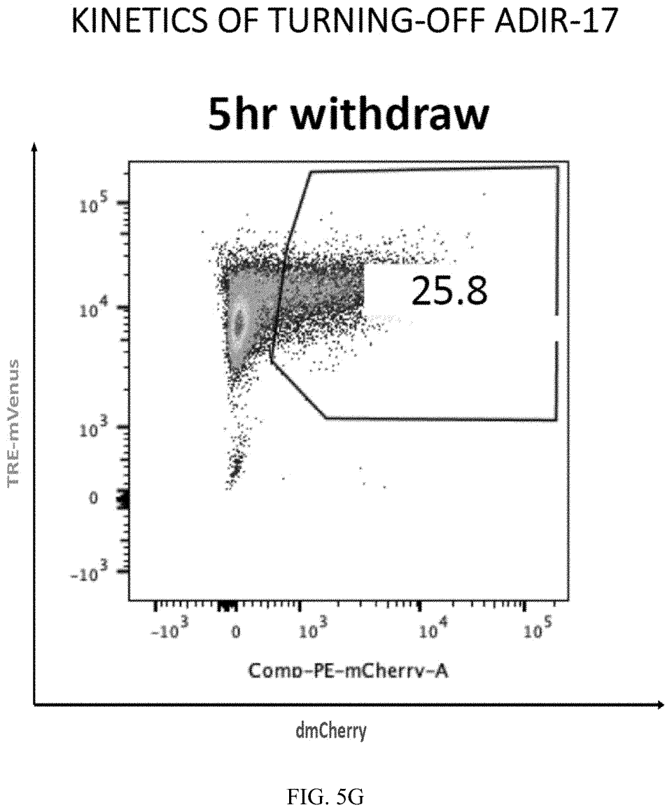

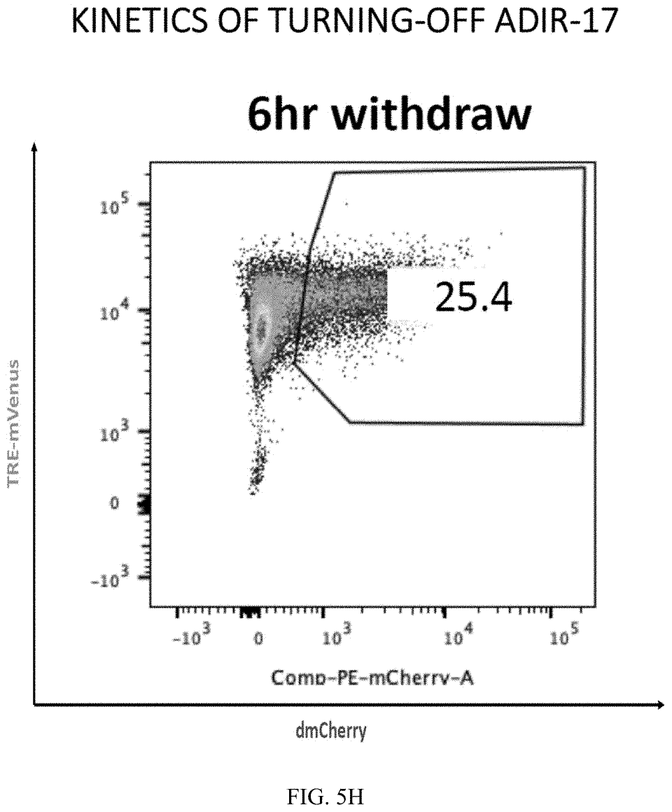

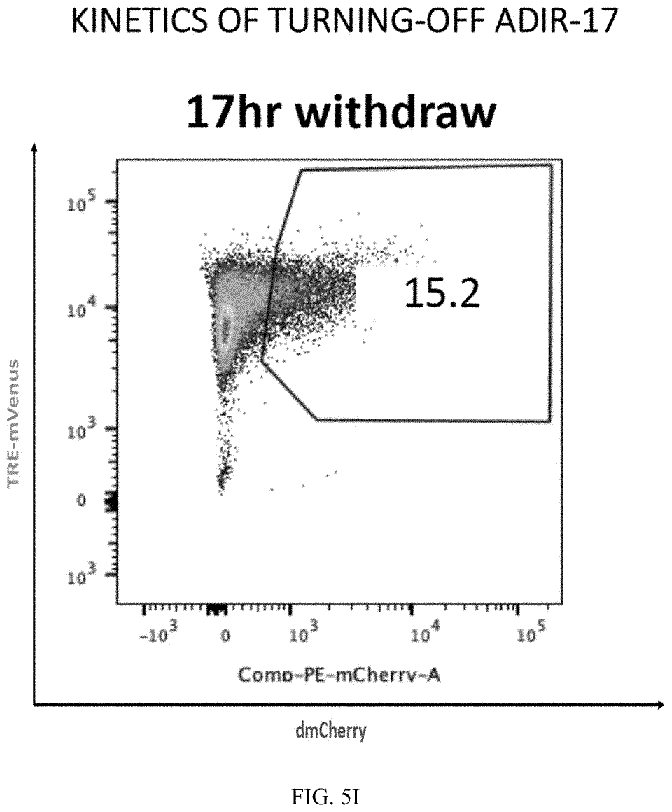

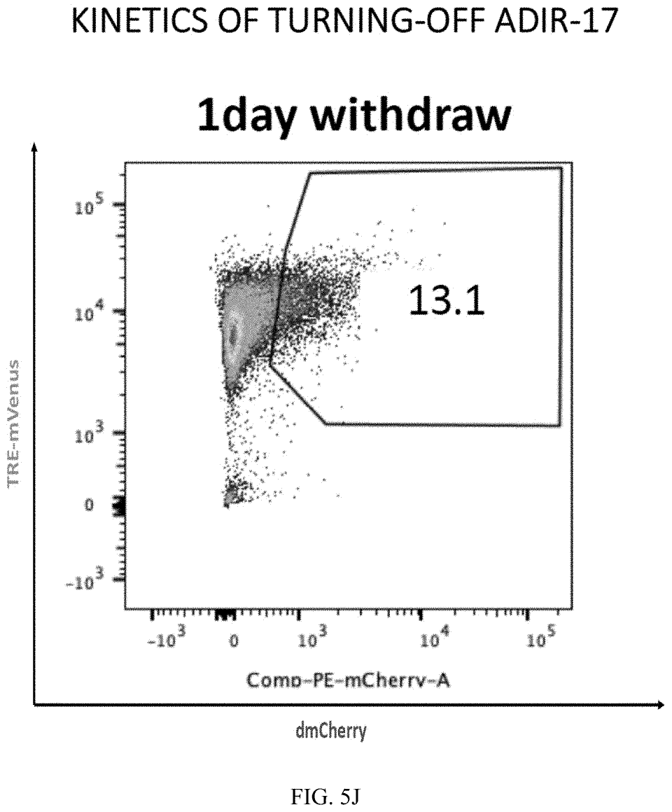

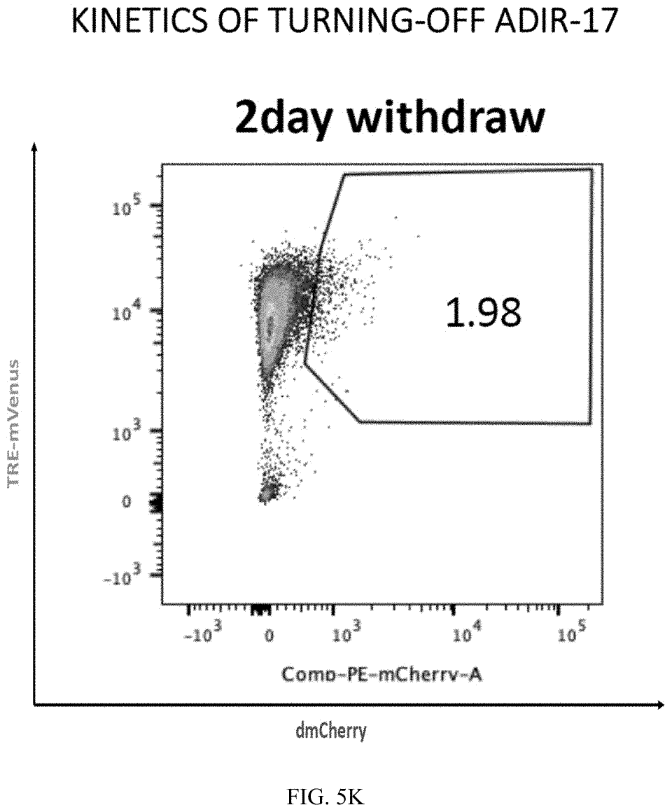

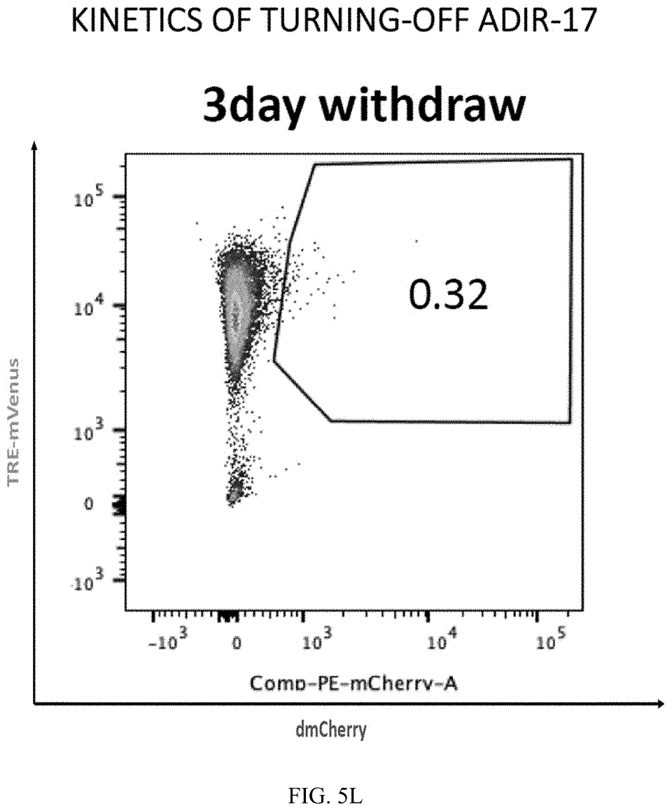

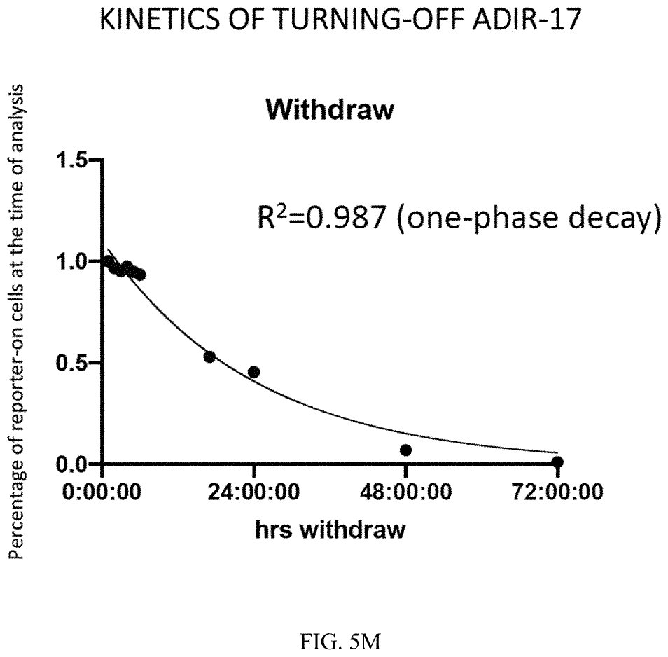

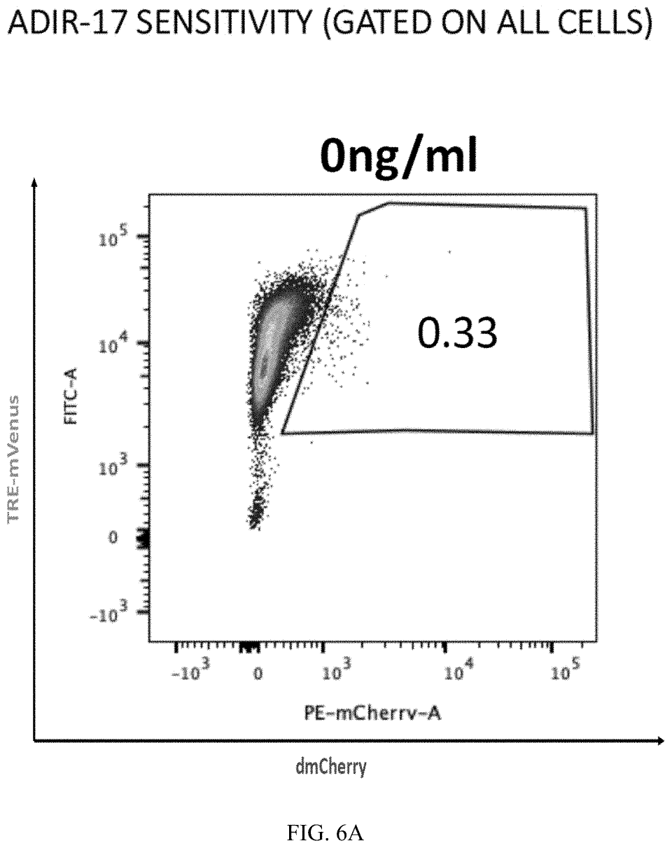

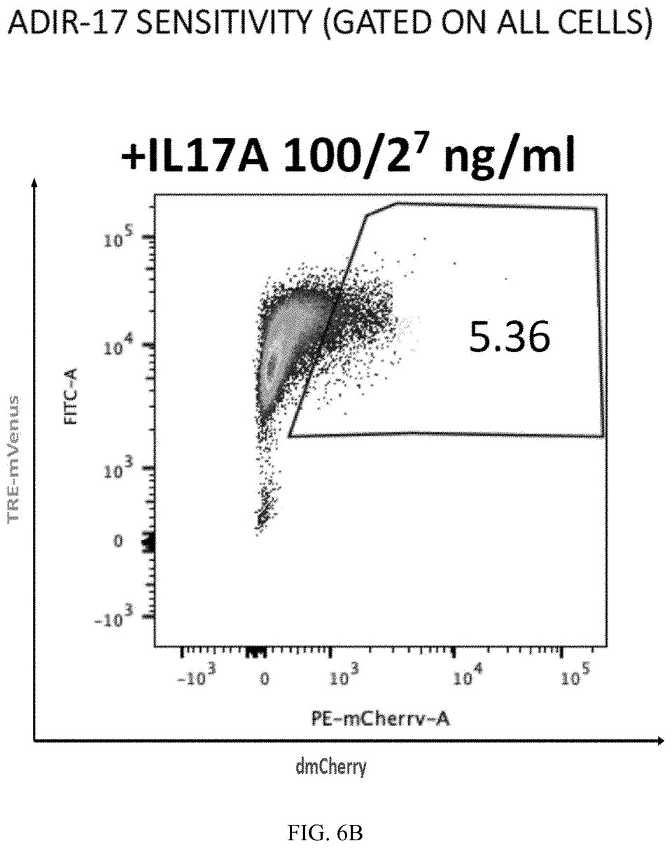

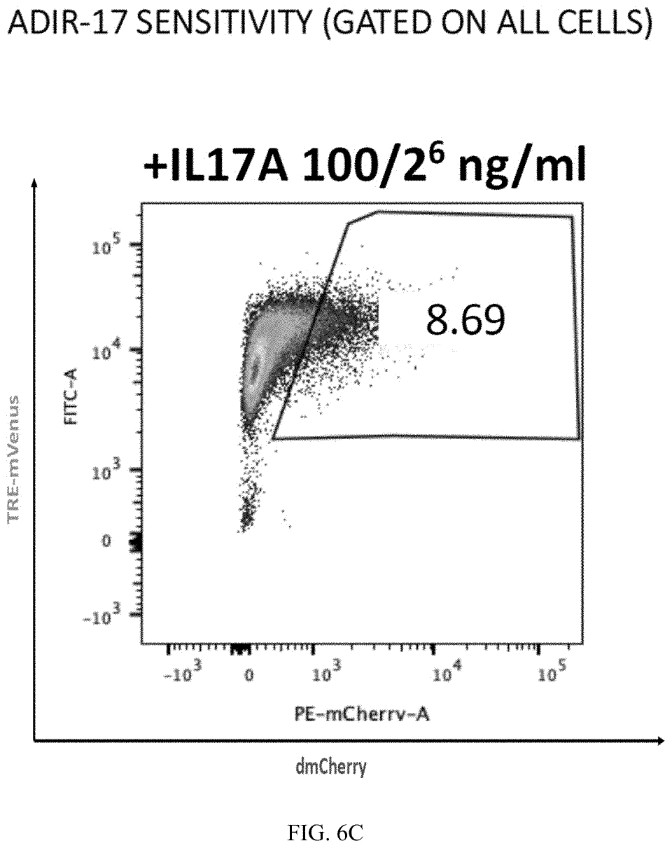

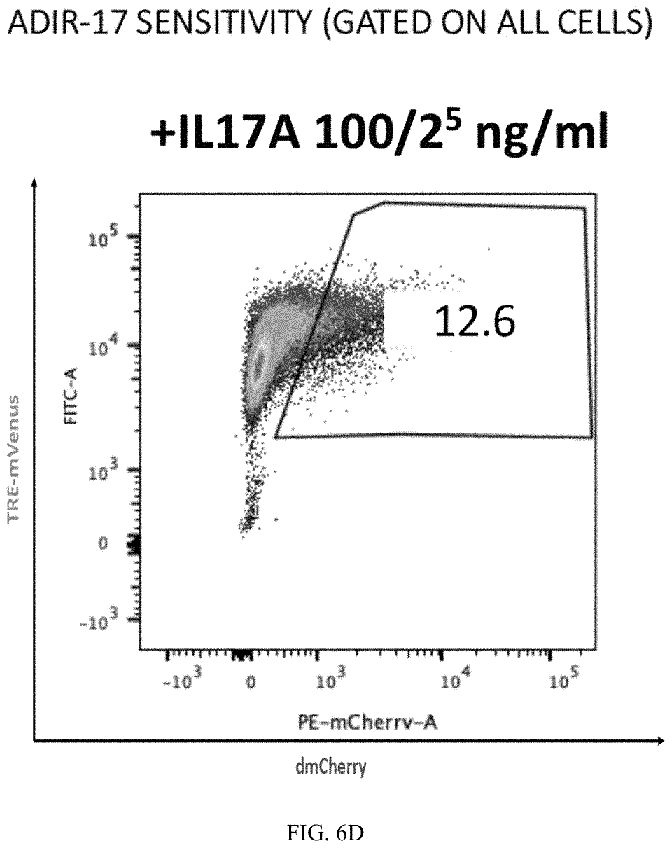

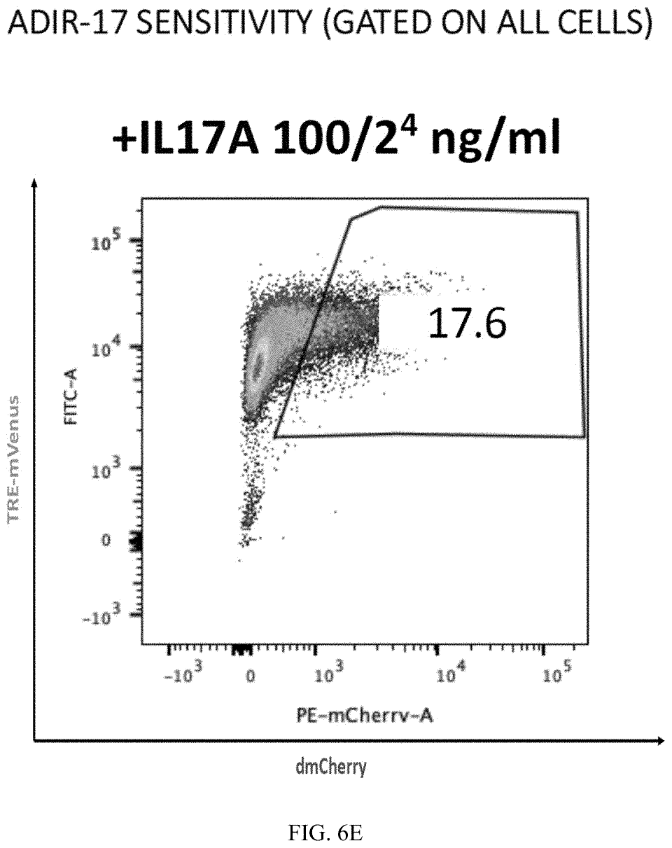

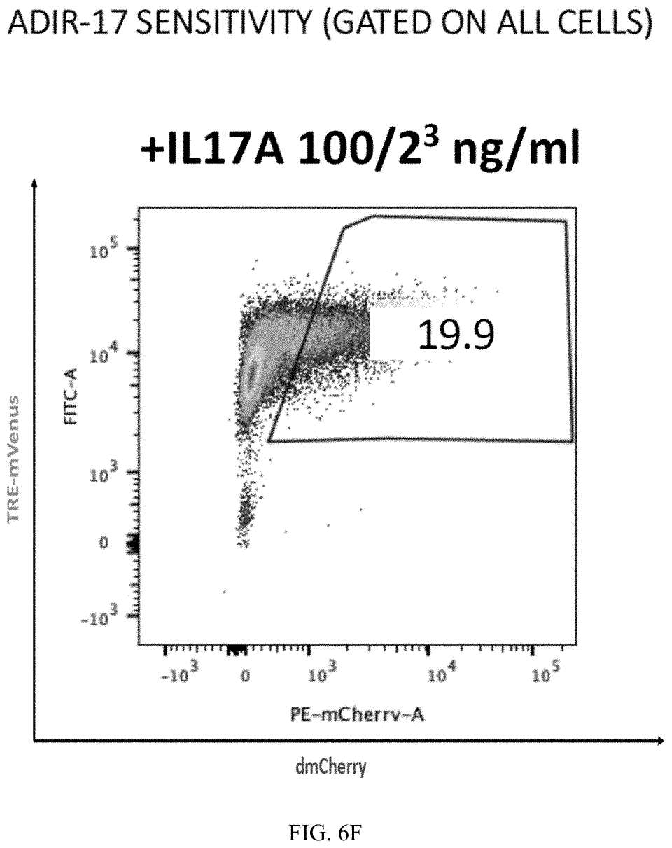

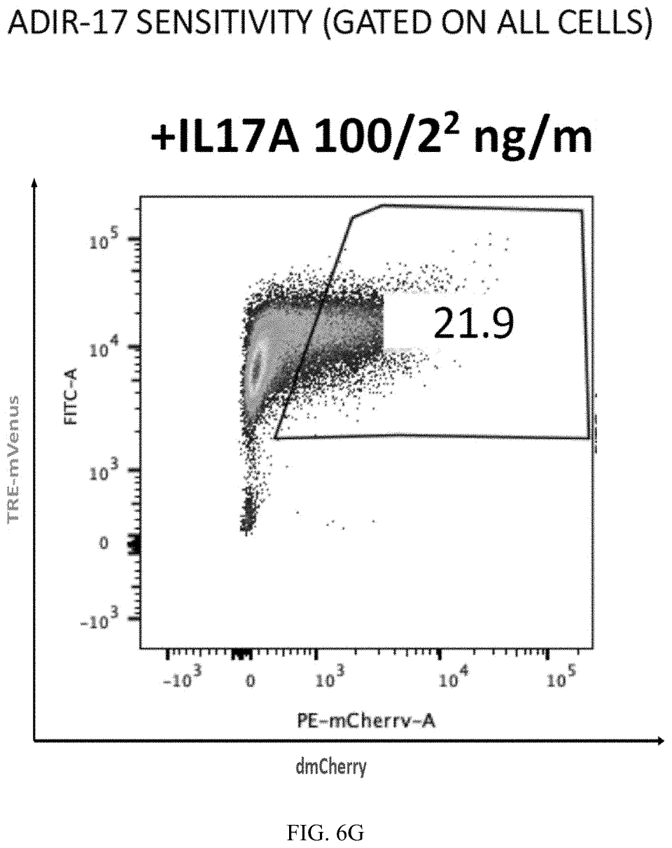

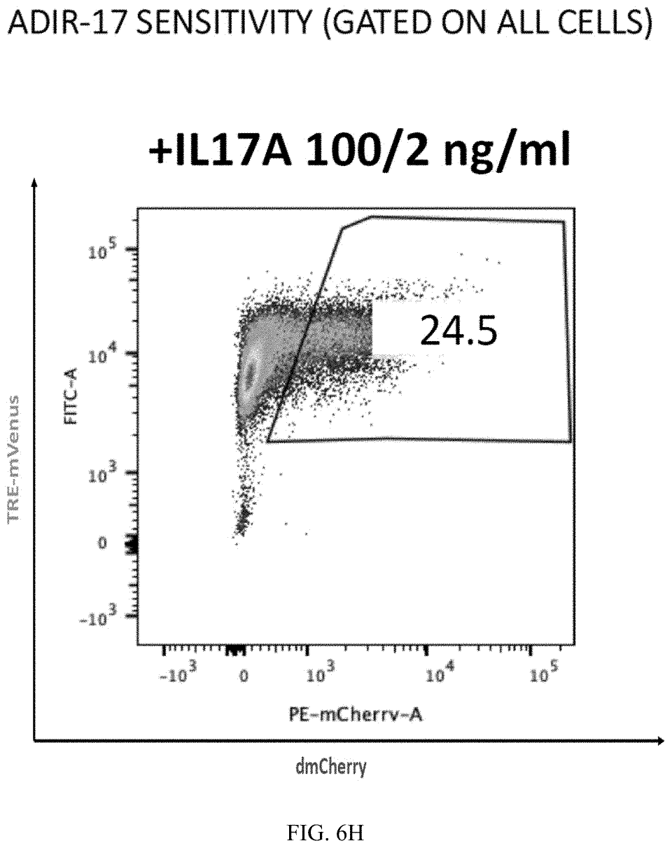

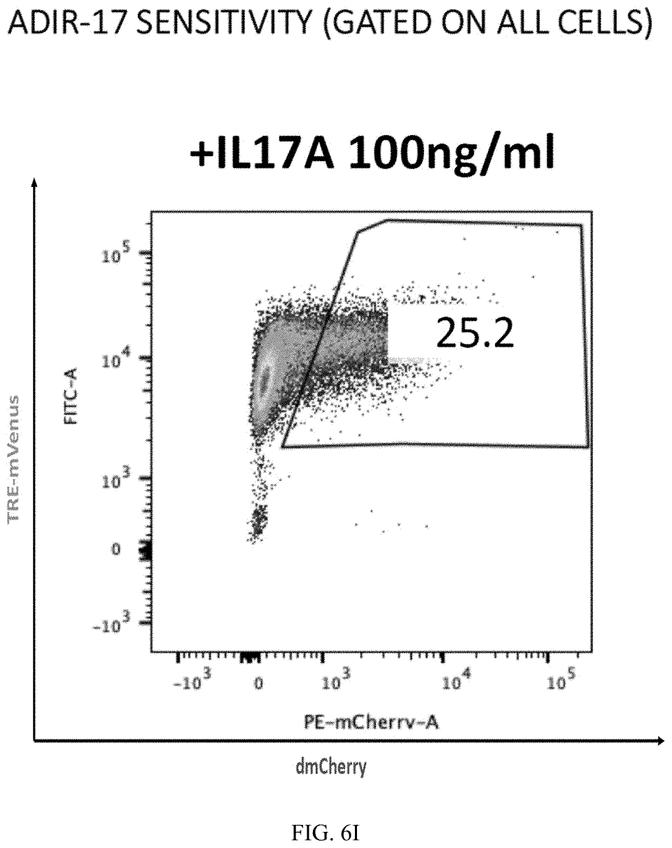

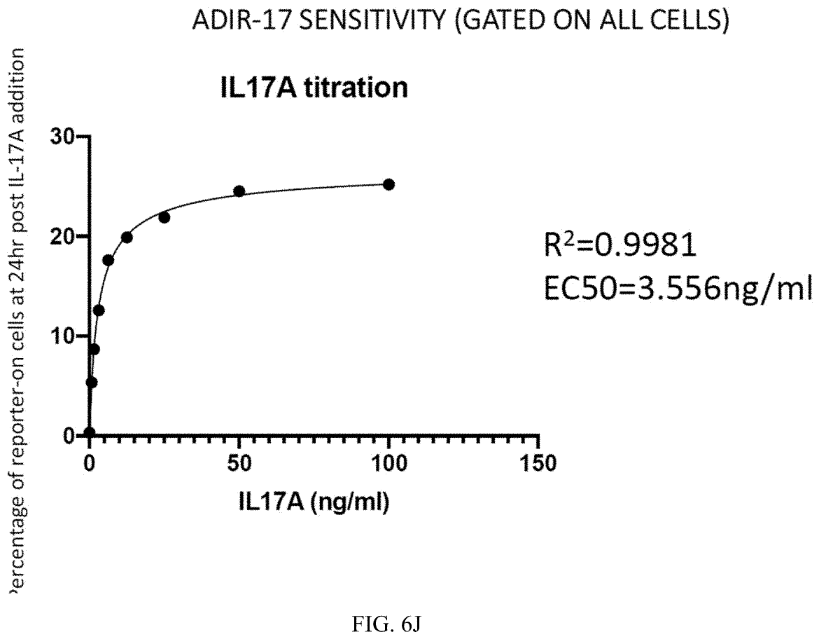

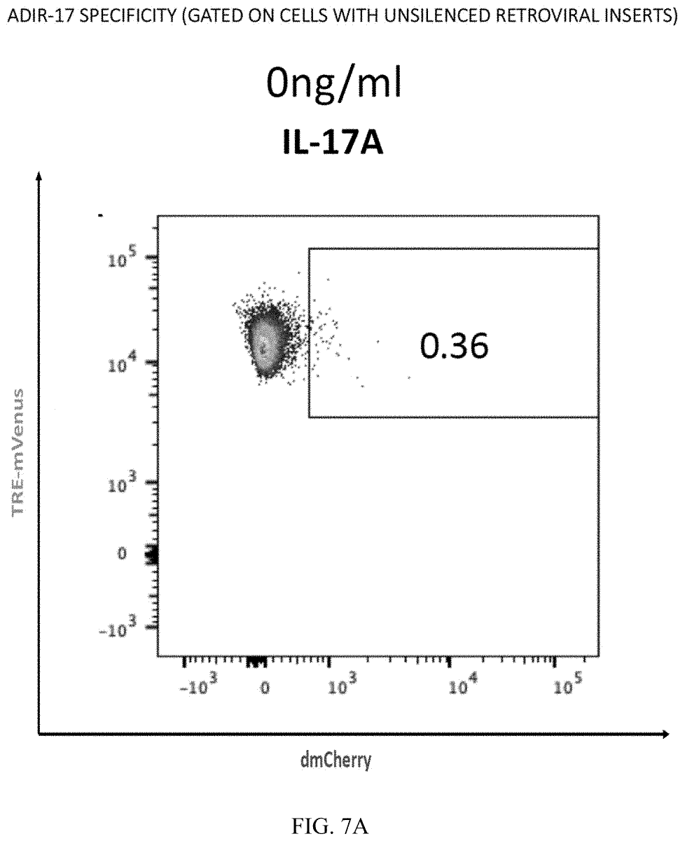

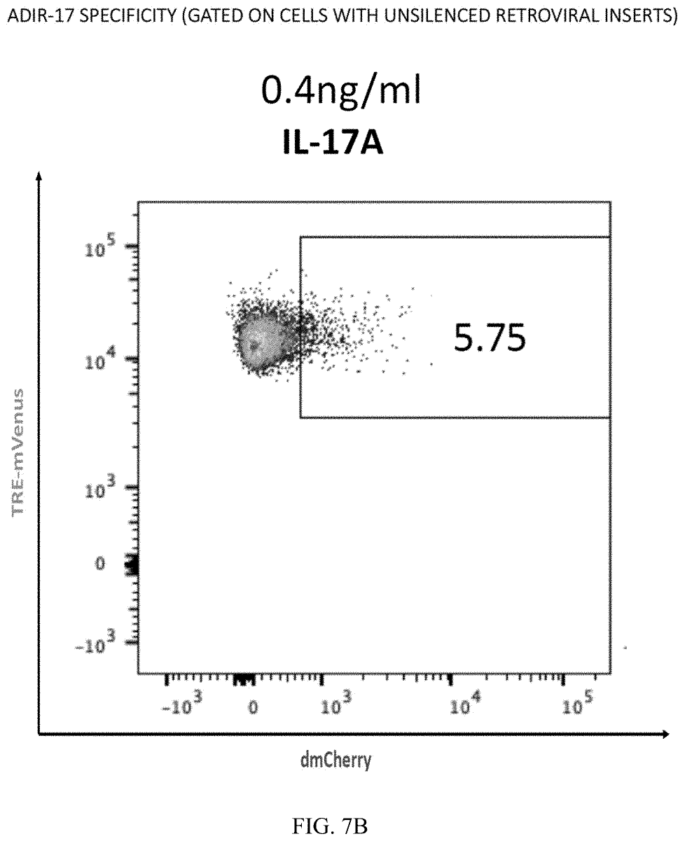

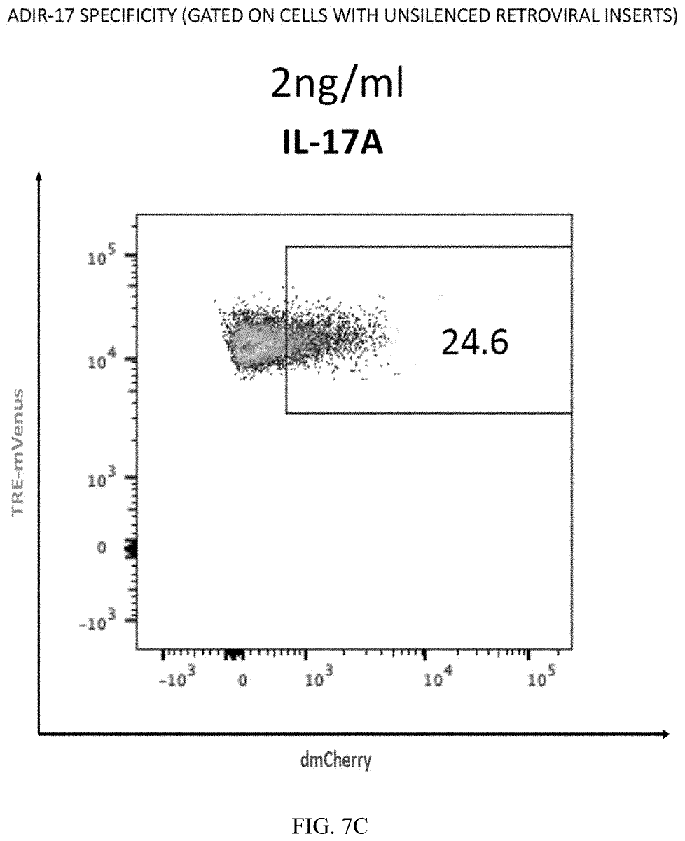

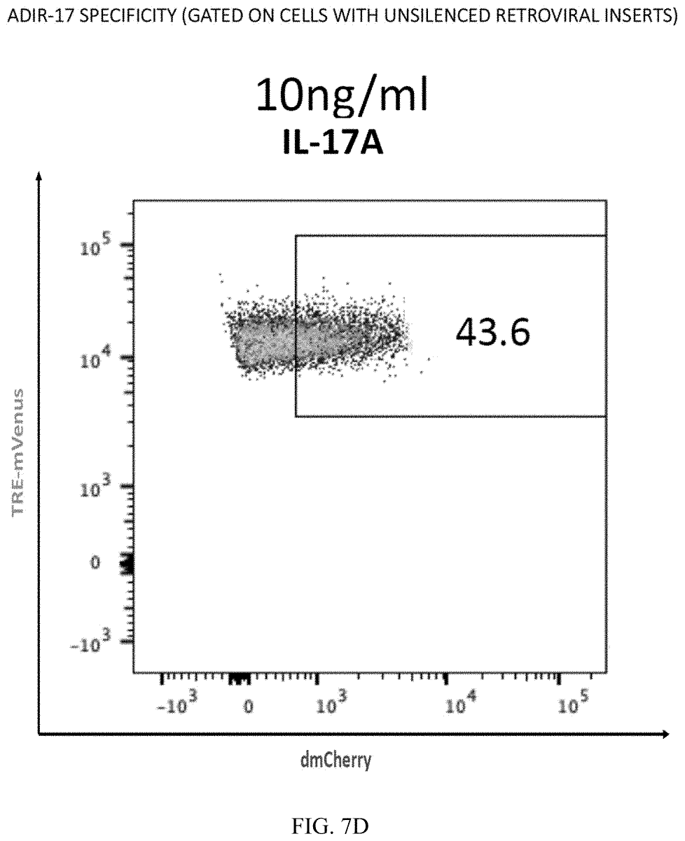

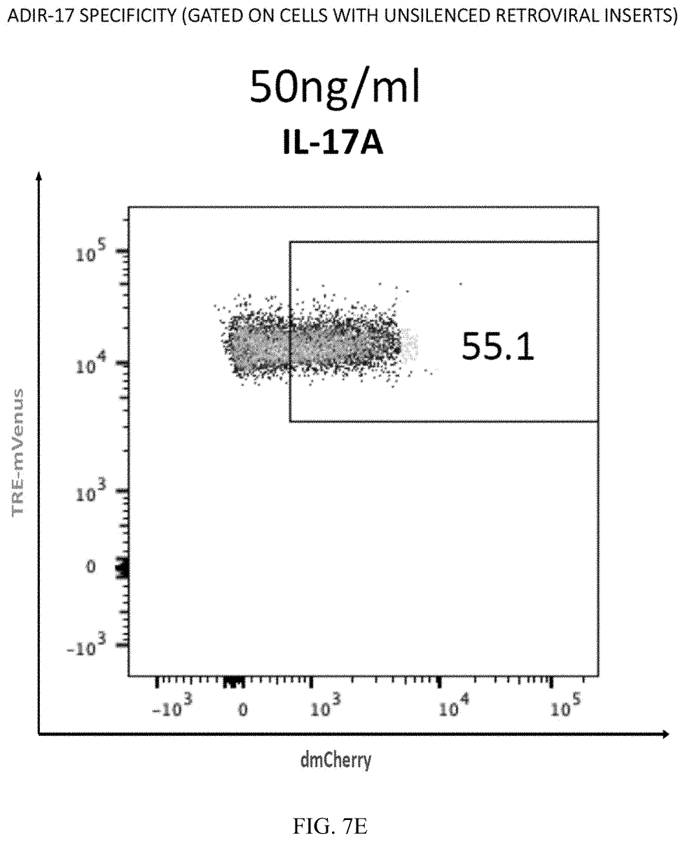

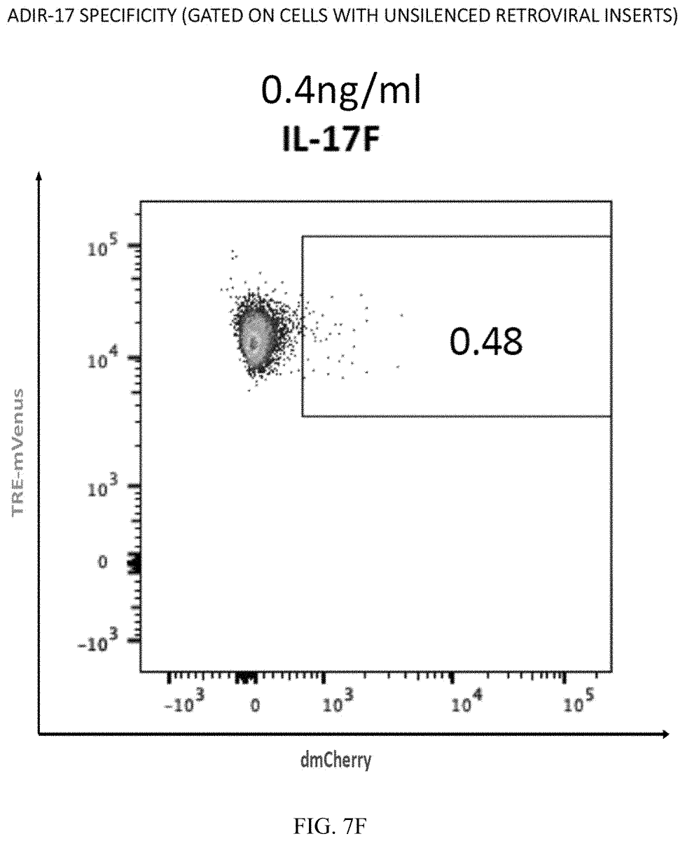

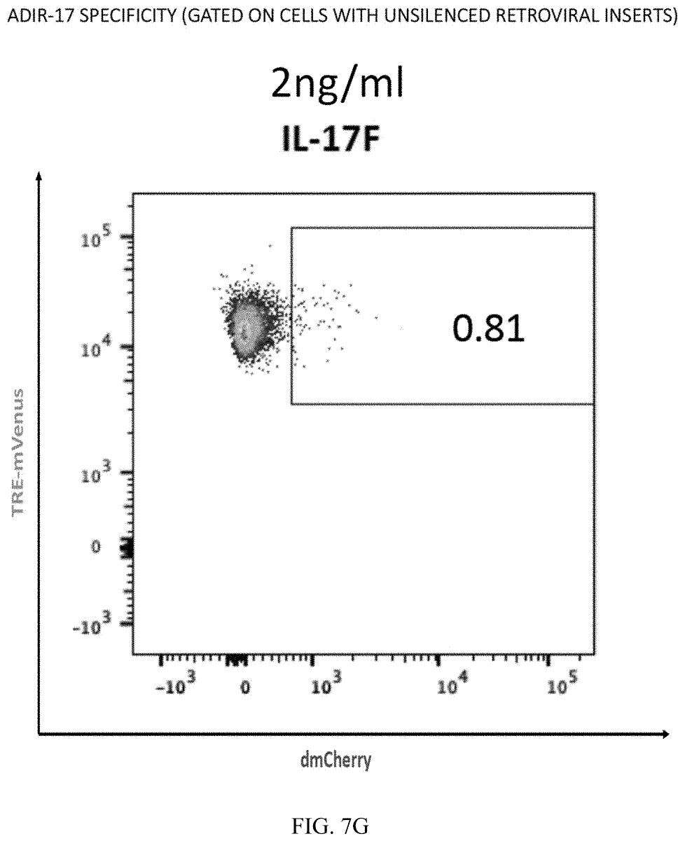

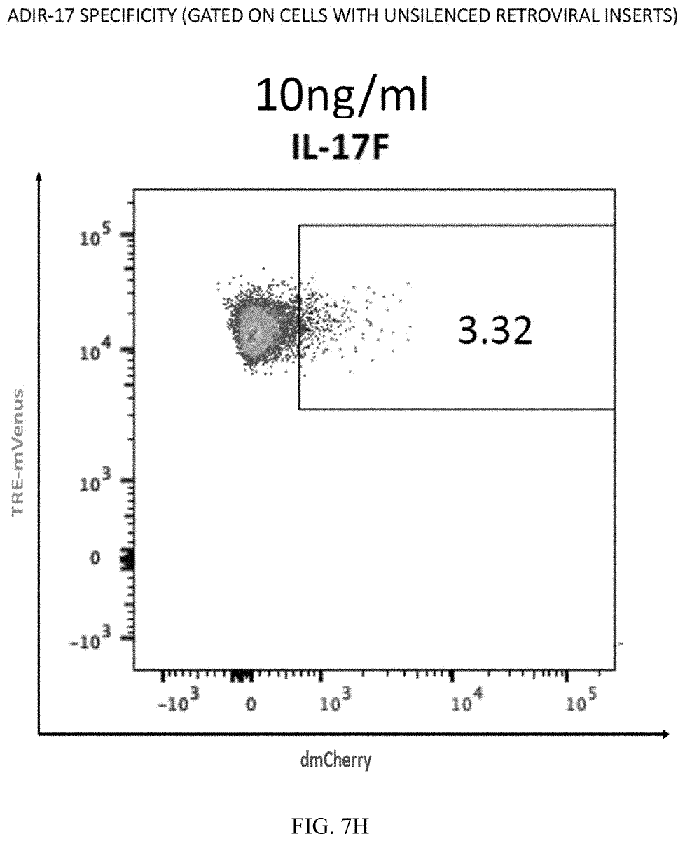

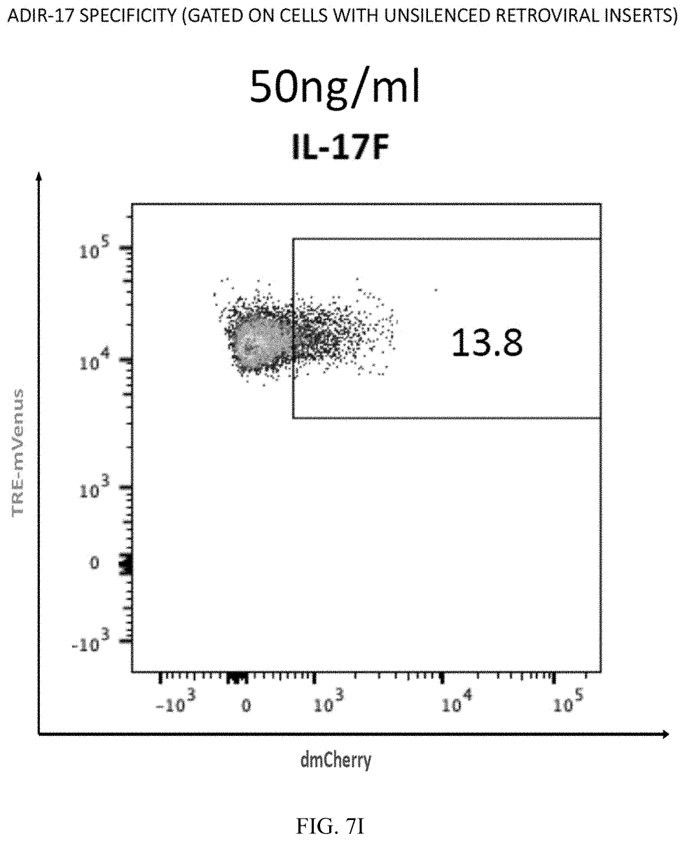

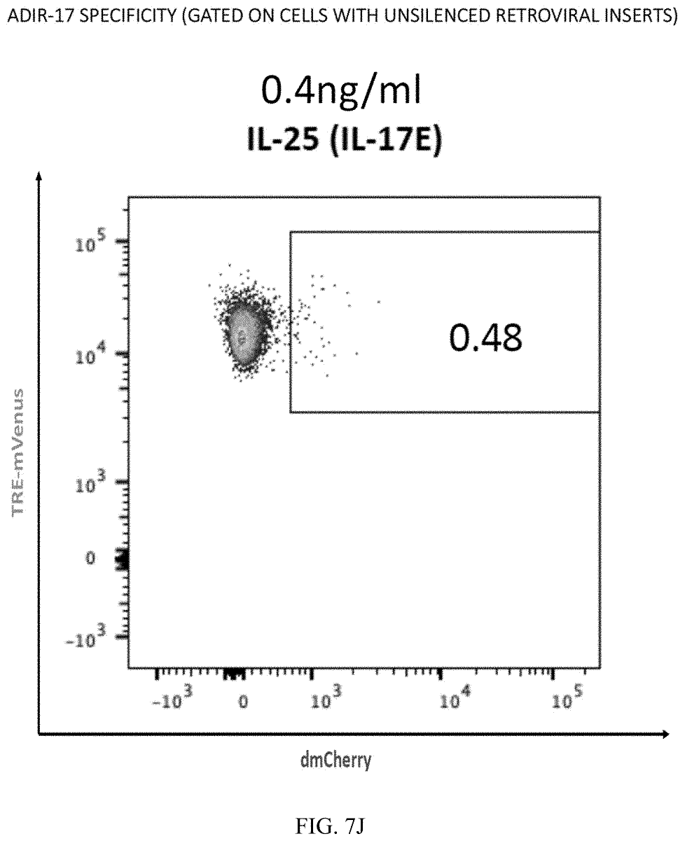

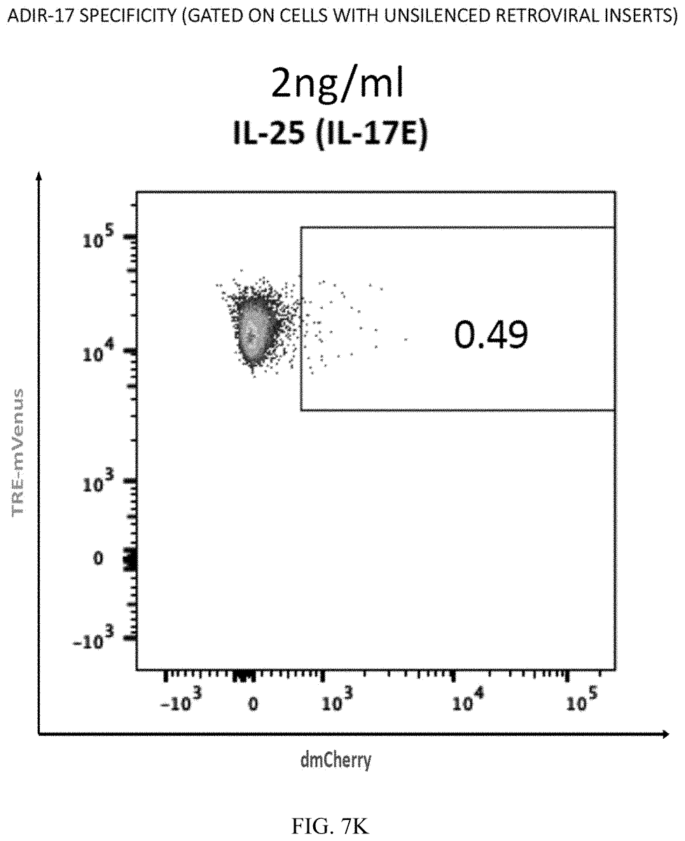

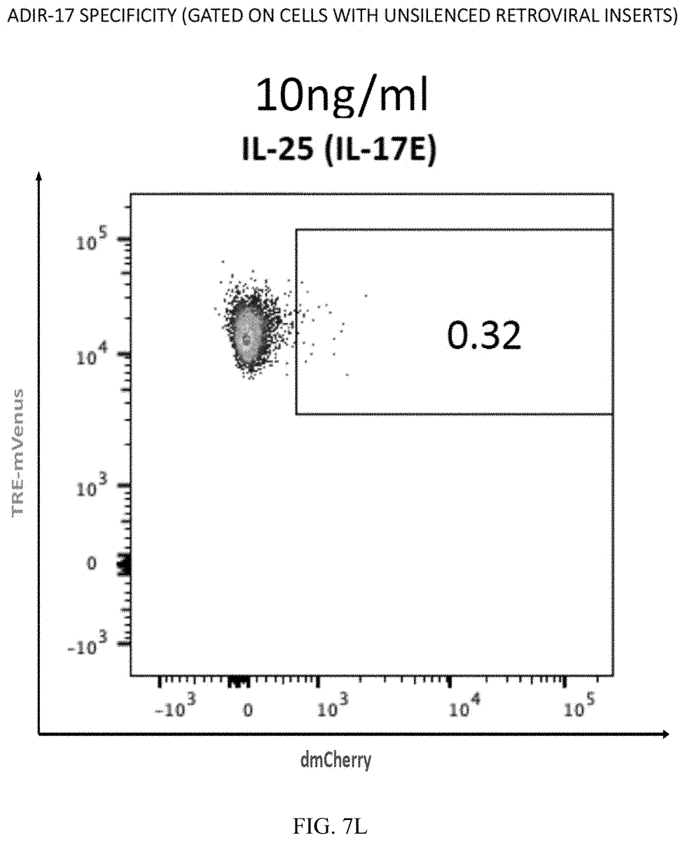

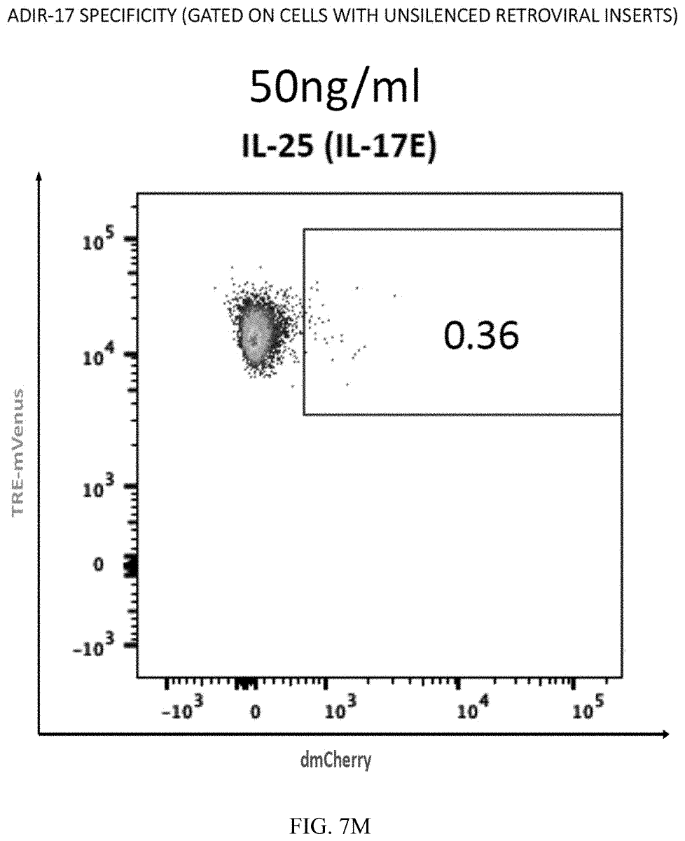

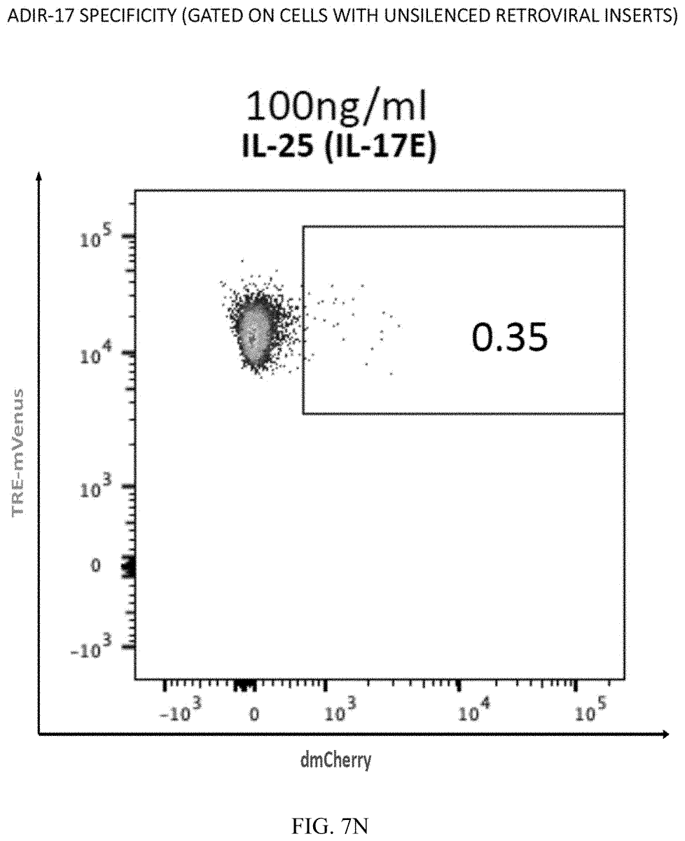

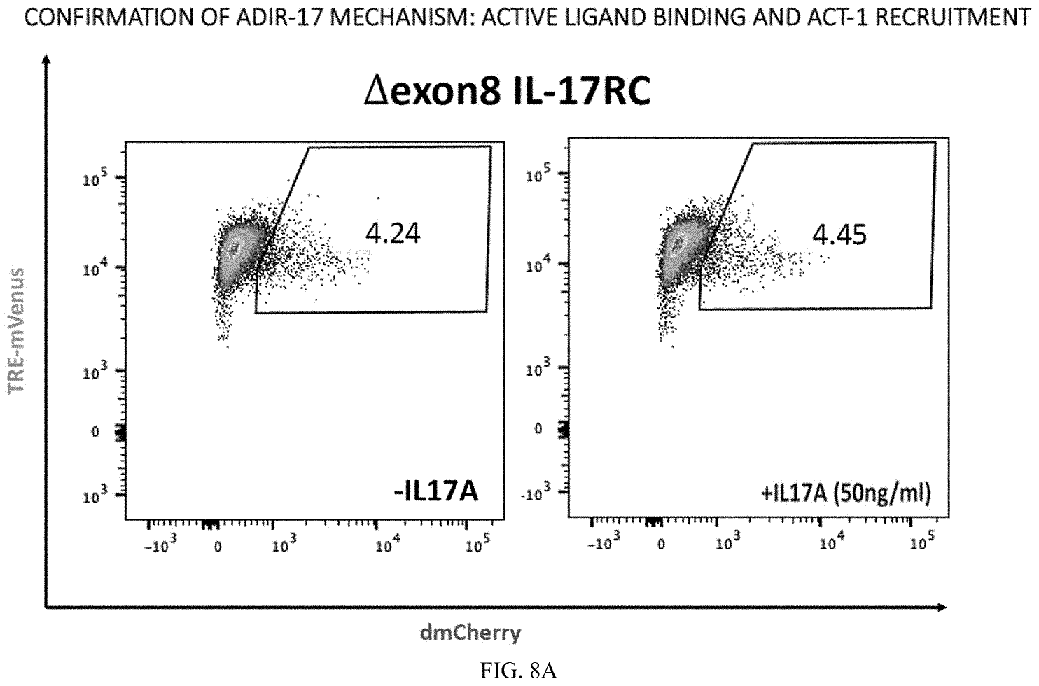

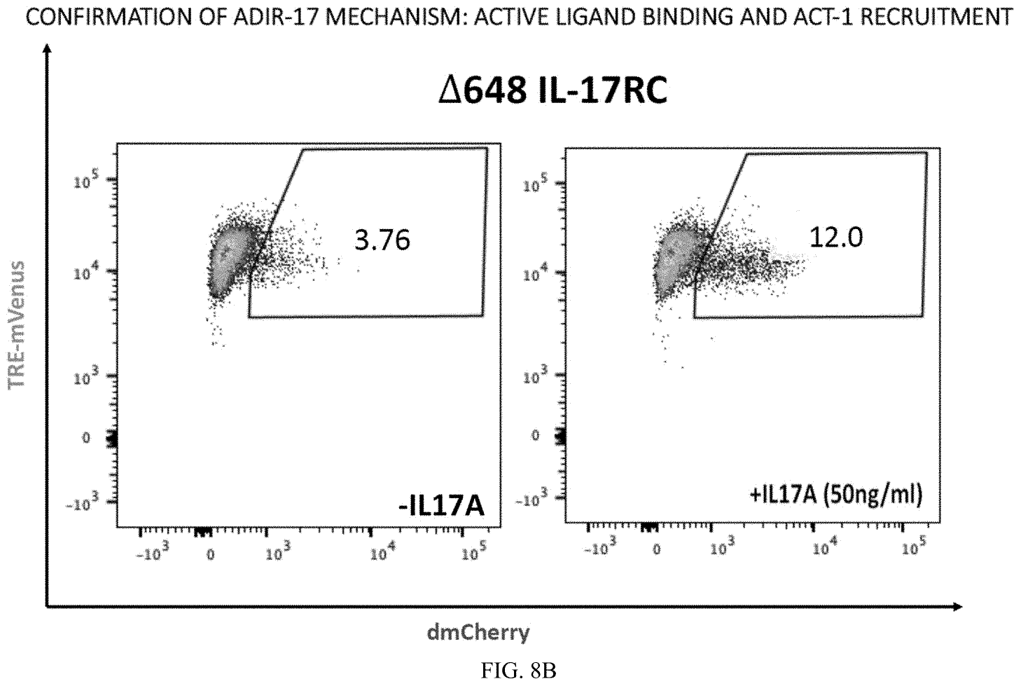

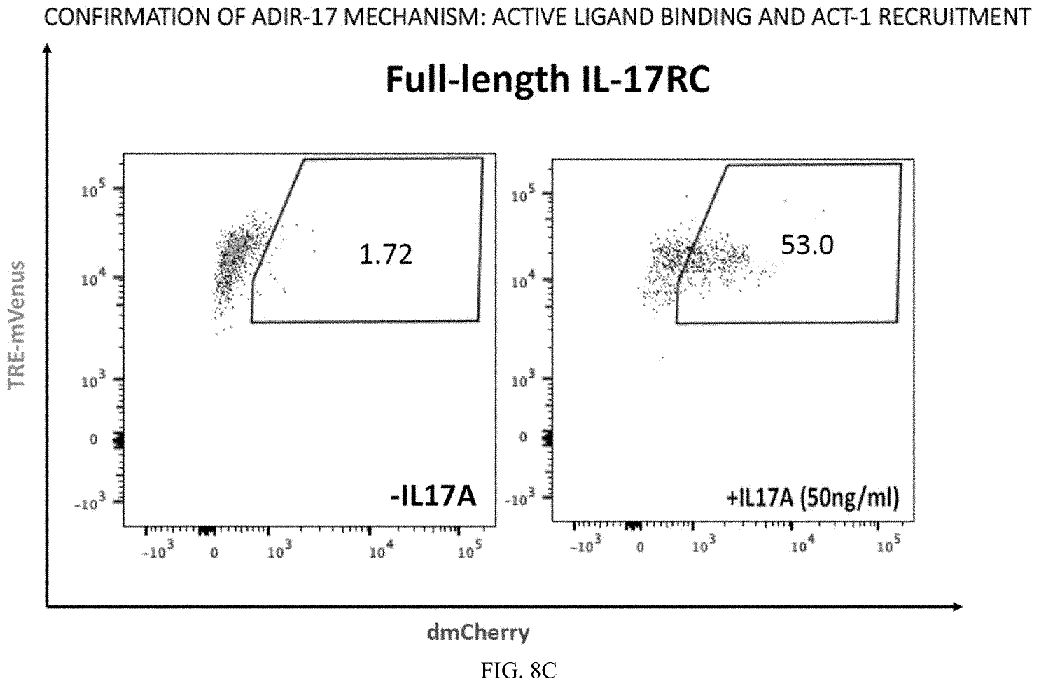

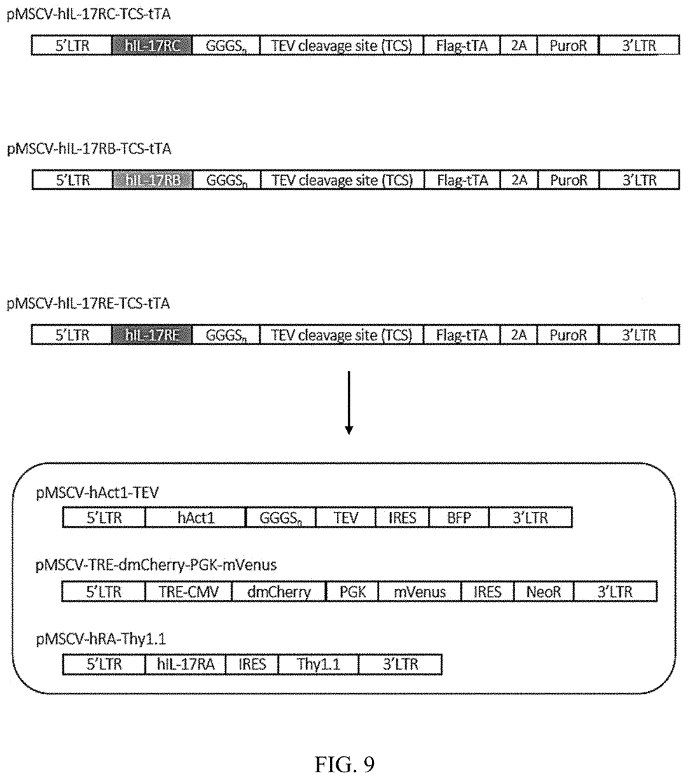

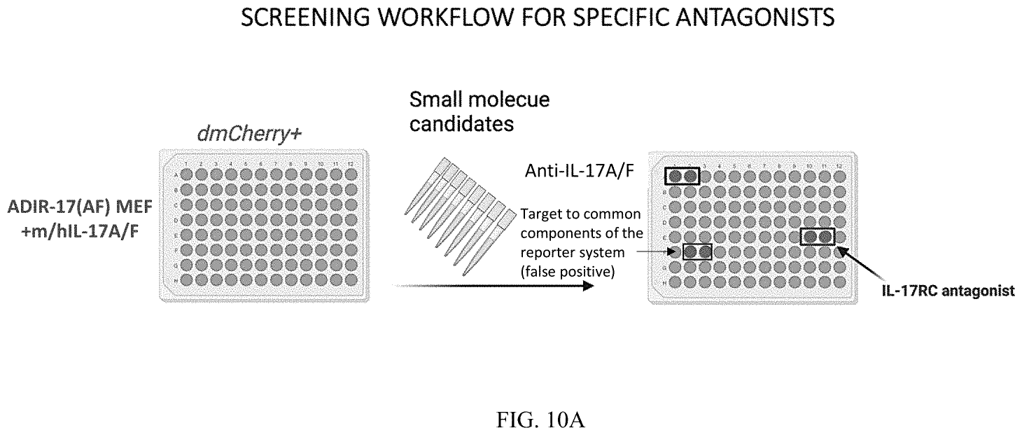

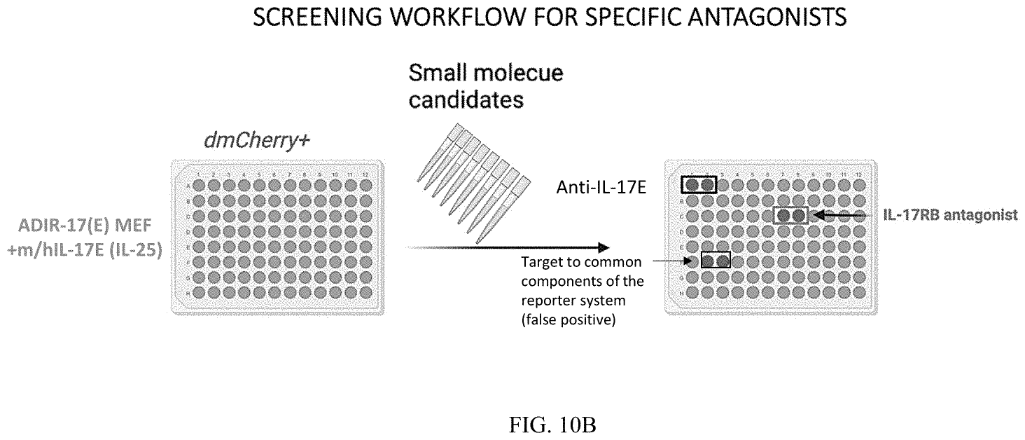

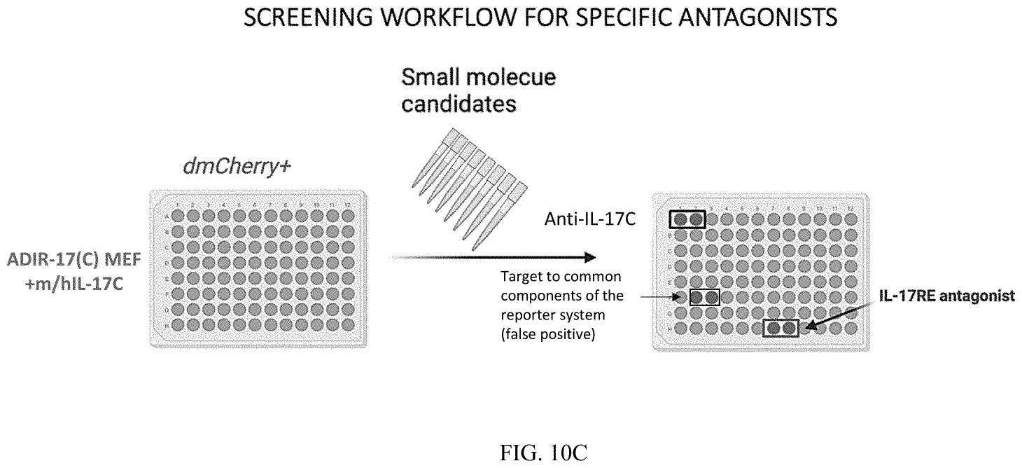

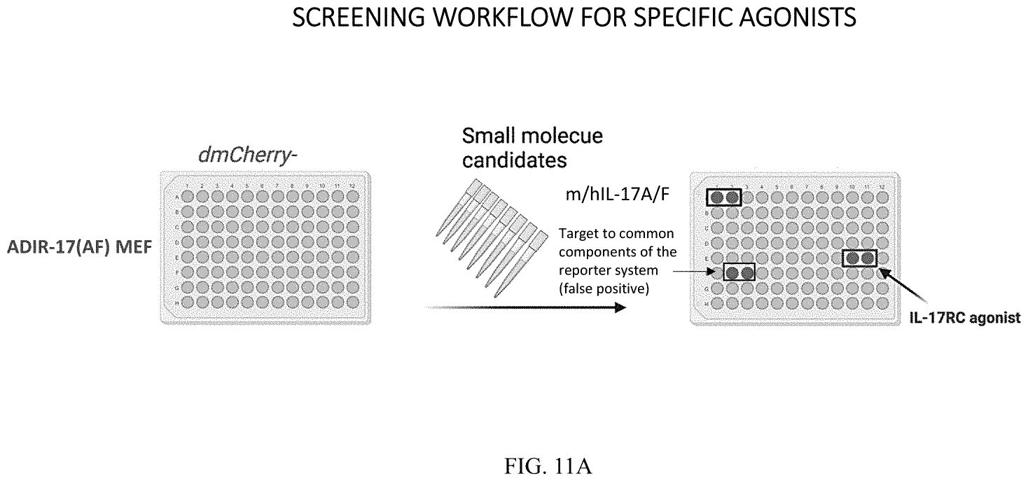

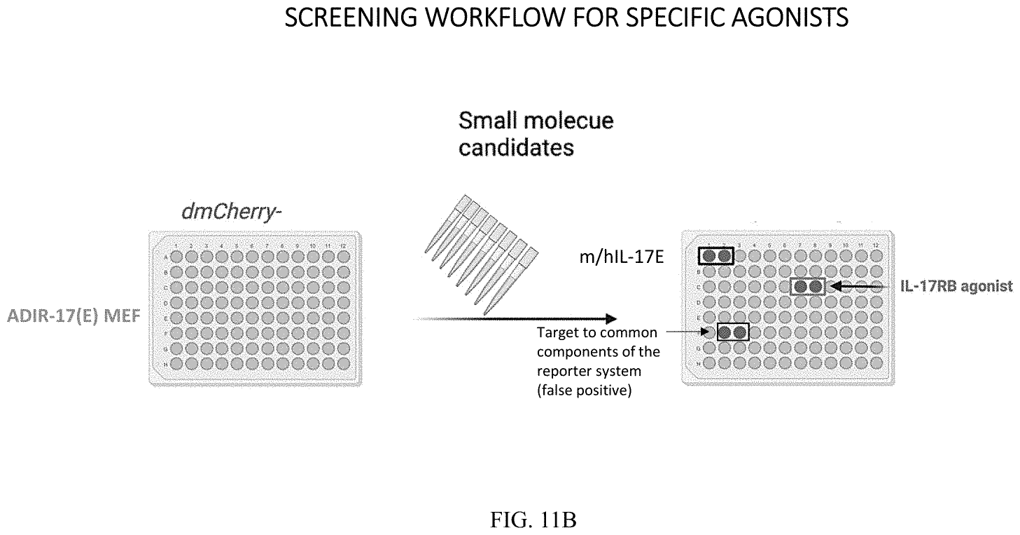

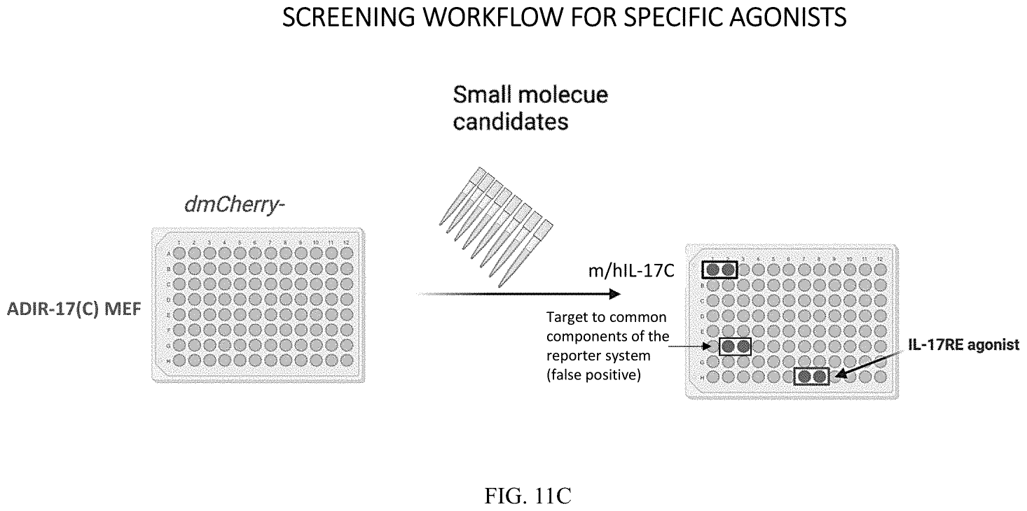

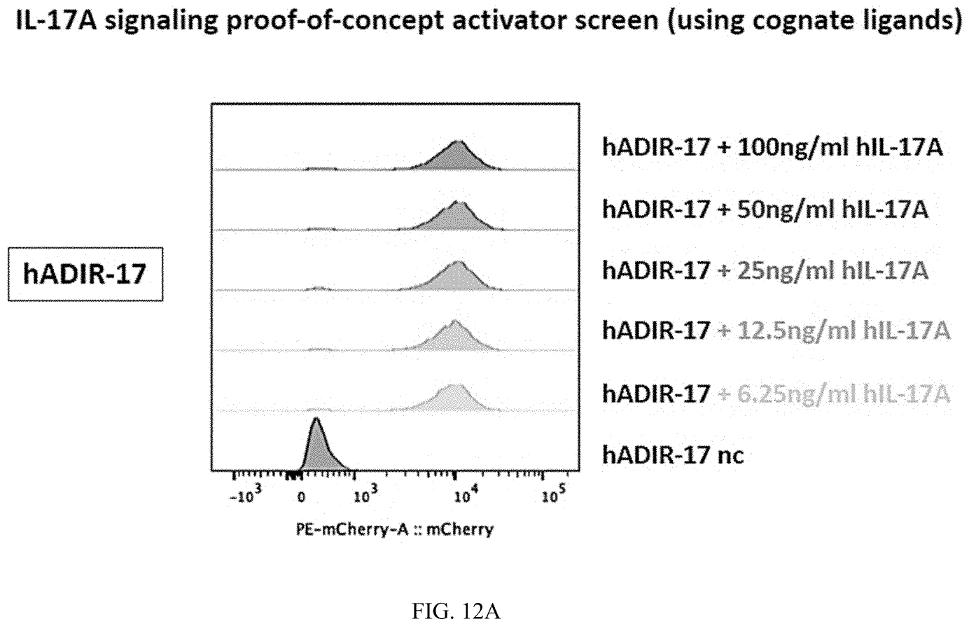

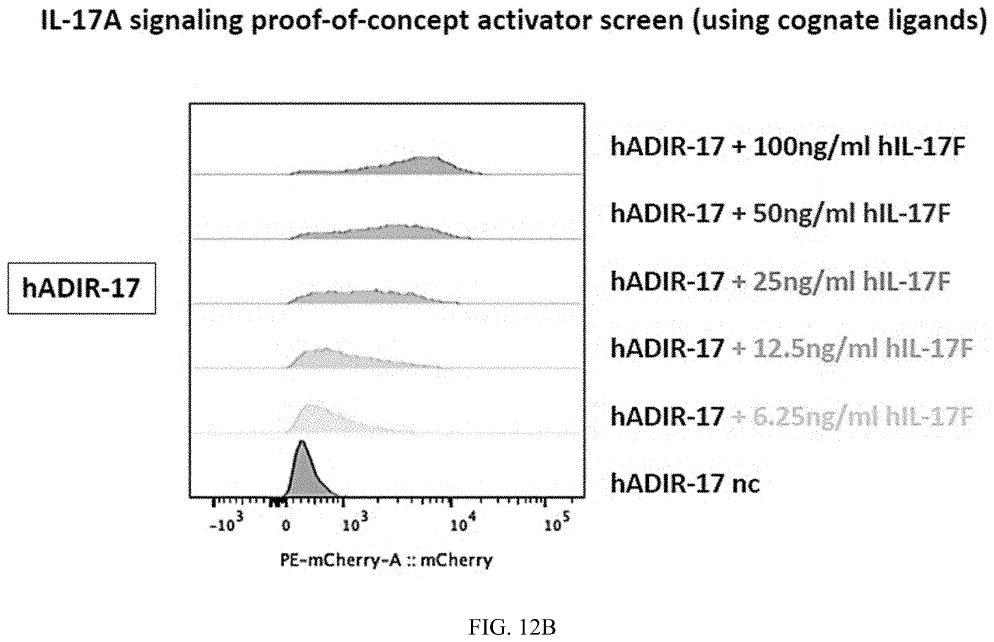

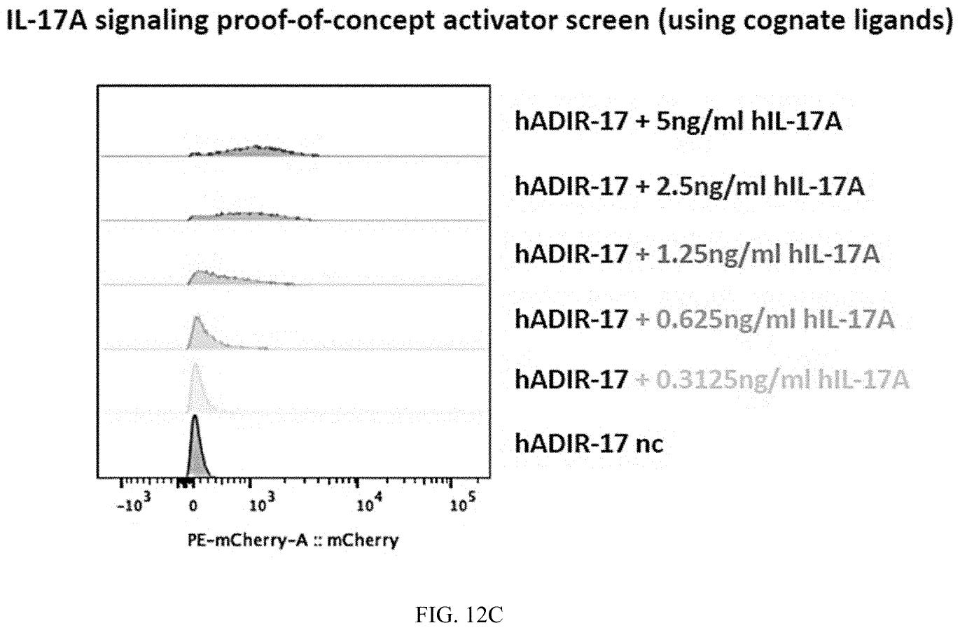

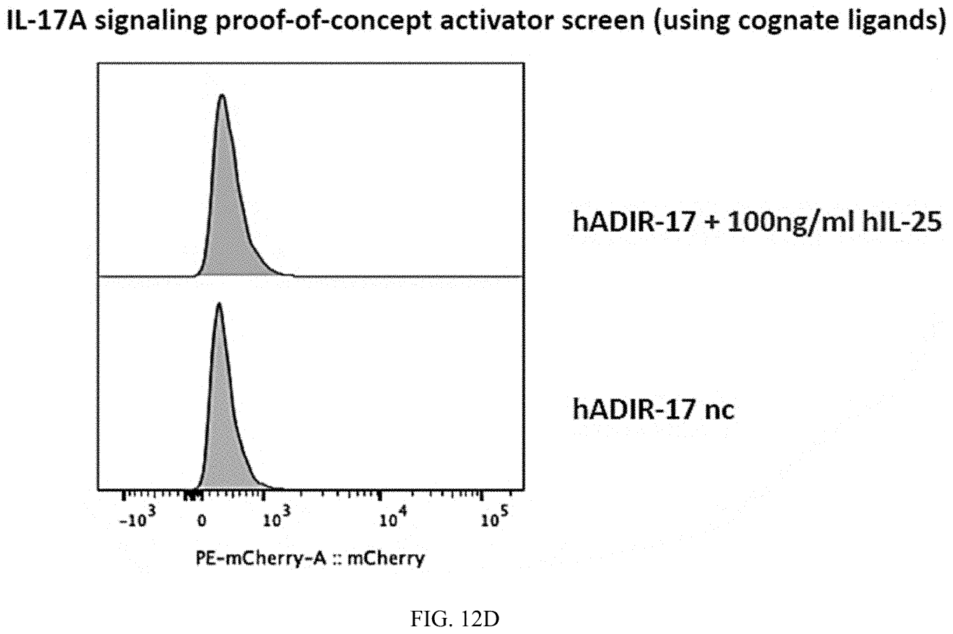

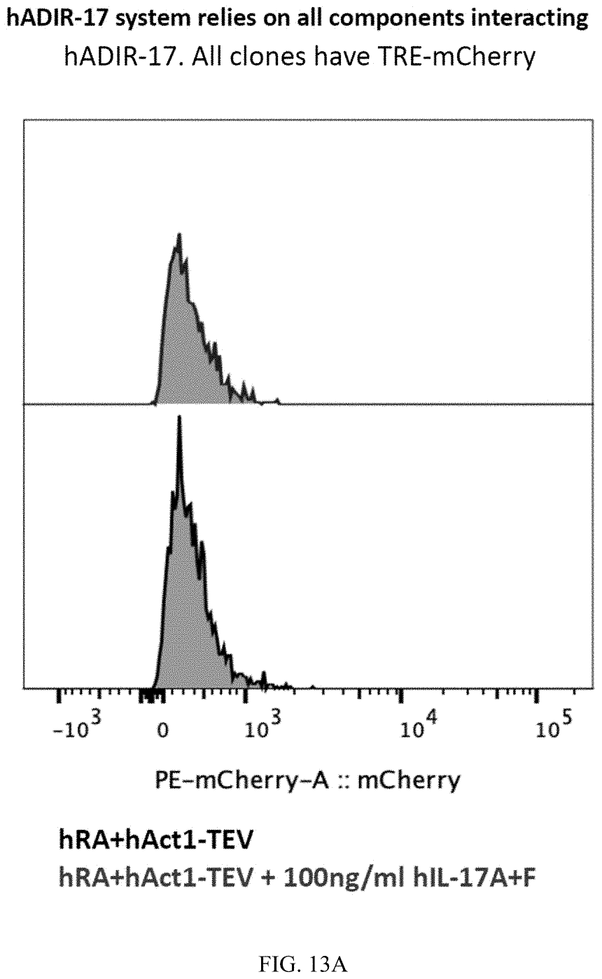

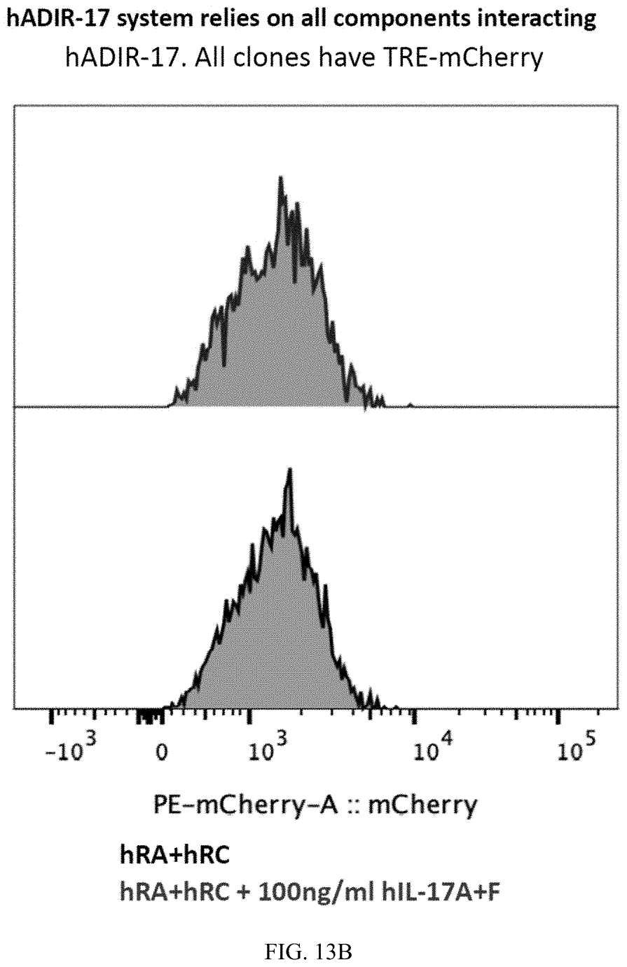

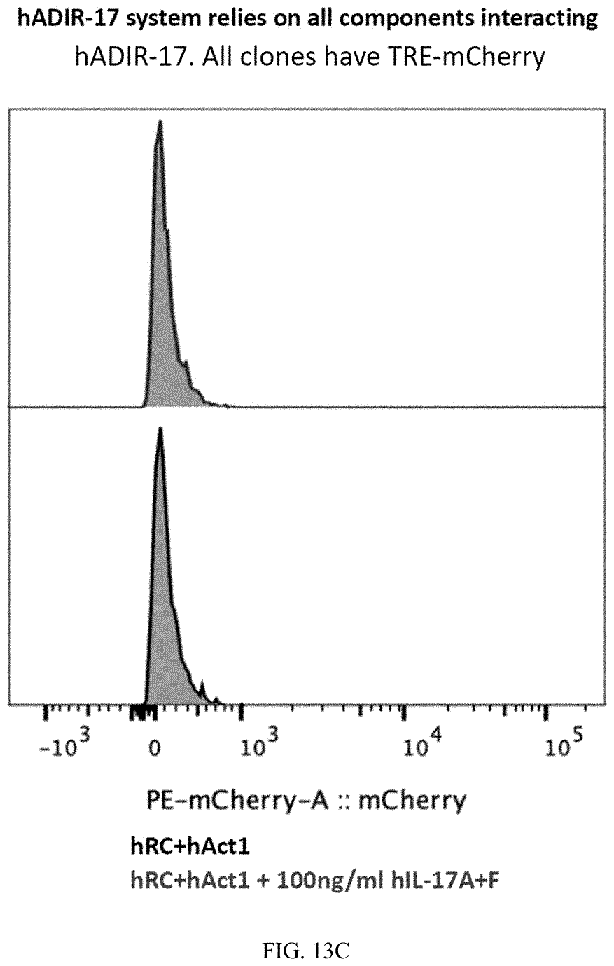

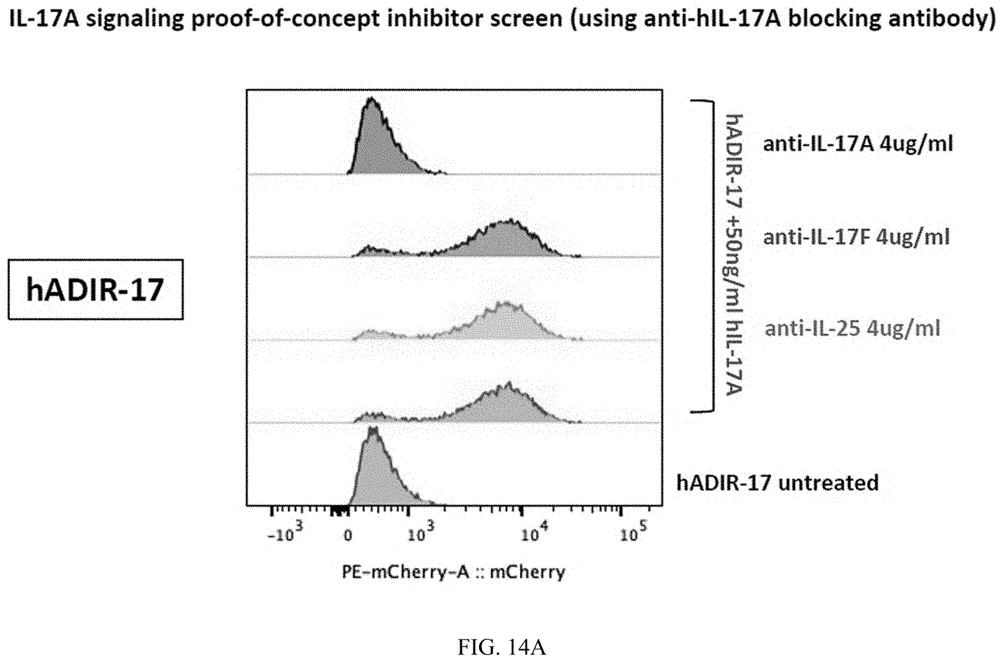

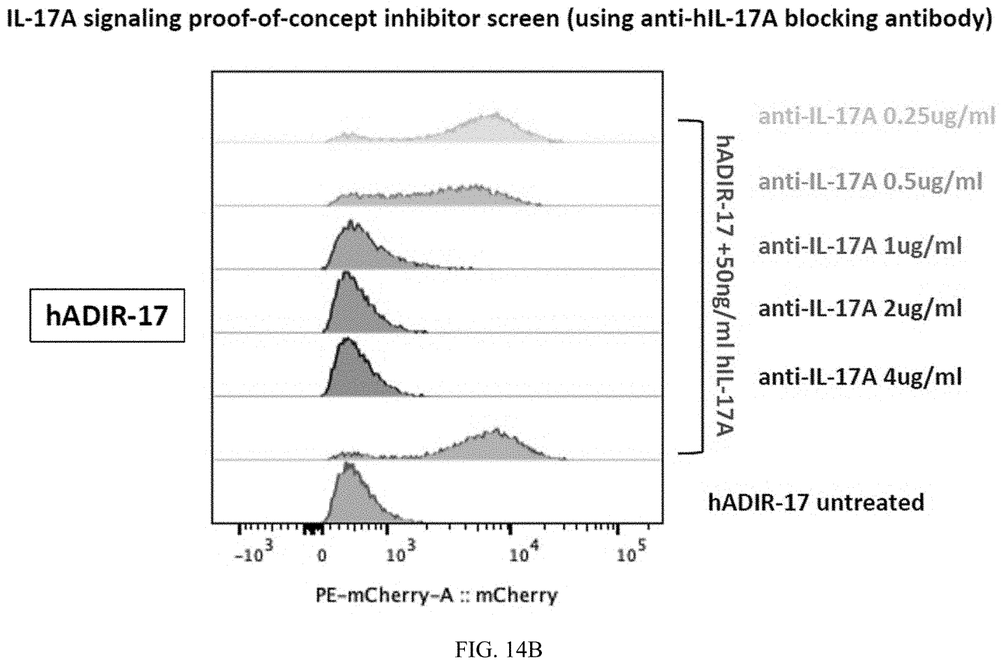

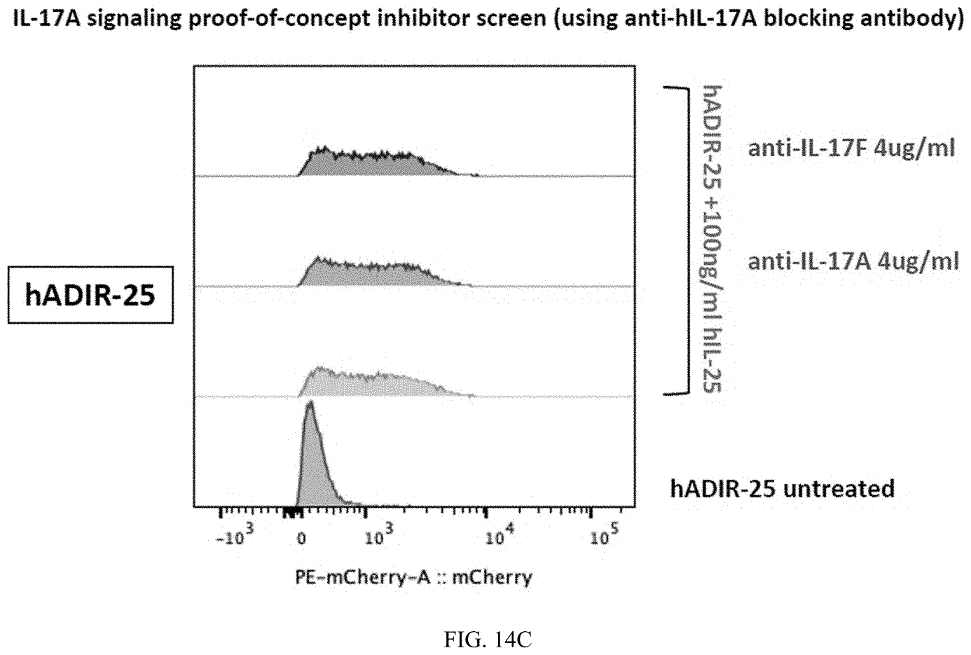

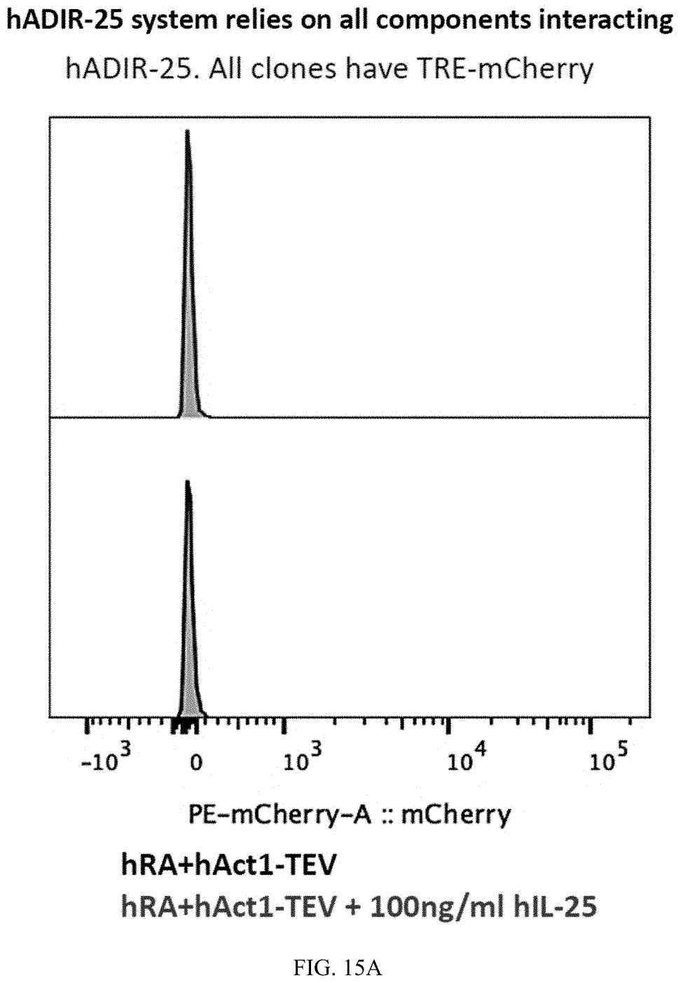

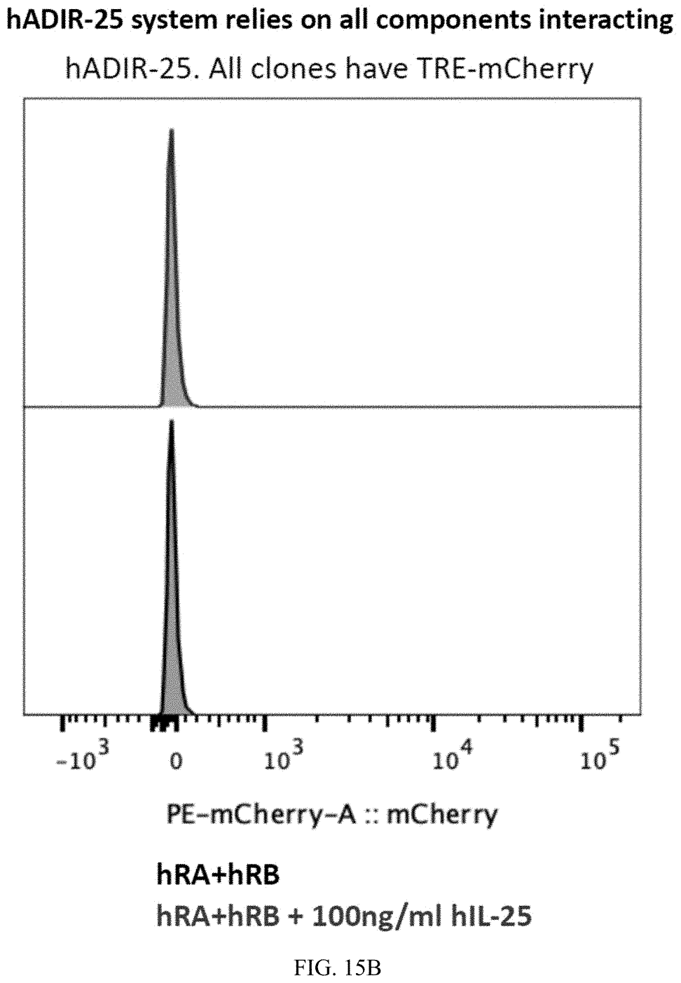

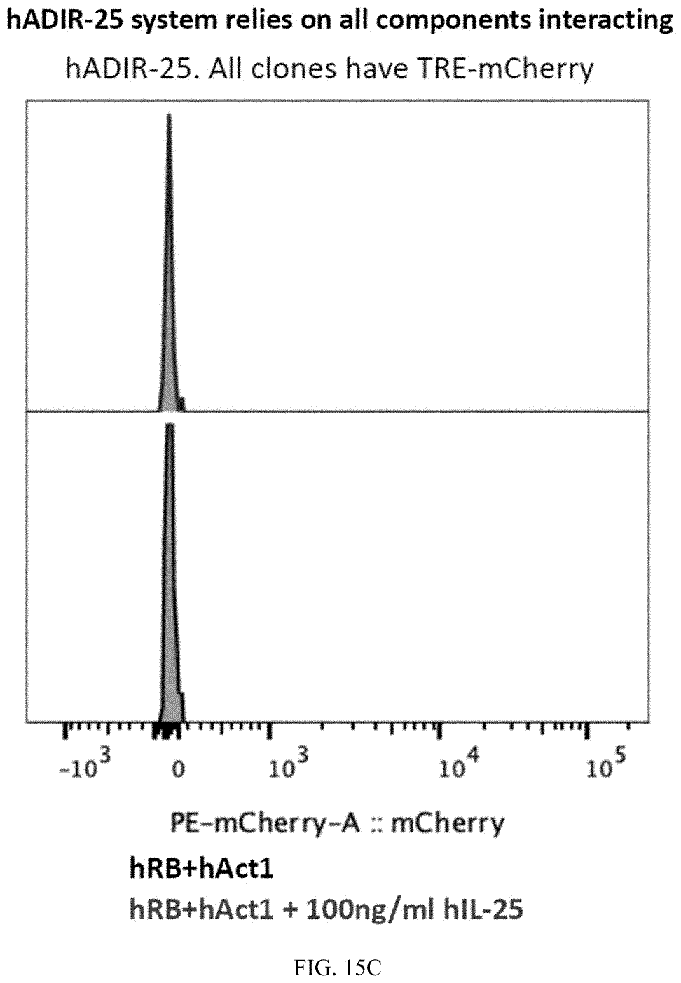

Mouse constructs were produced as depicted in the panels of FIG. 1. Human sequence constructs were produced as in the panels of FIG. 9, where mouse cDNA constructs were replaced by human cDNA constructed. In the mouse ADIR system exogenous IL-17RC was not provided because the cell line (mouse embryonic fibroblasts) have endogenous IL-17RC expression. In the hADIR system, human components used for the hADIR system (hRA, hRC, hRB, hAct1) were expressed from added polynucleotides.

The data reflected in the Figures utilized a reporter system specifically designed for Act1-dependent IL-17 signaling pathways. The reporter system is based at least in part on the principle that the engagement of active cognate ligands, and the interaction between Act1 and IL-17 receptors is required for active signaling of IL-17A/F, IL-17E, and IL-17C. The protein-protein-interaction (PPI) based reporter system is highly specific and easily scalable. The disclosure is illustrated using mouse and human systems. For the data presented in the Figures, the system comprised a three-part reporter system specifically designed for detecting IL-17A/F, IL-17E, and IL-17C signaling. The PPI-based reporter system provides for detecting active signaling, and is specific for each of the IL-17 family cytokines. The system that is responsive to binding of IL-17A and IL-17F comprises (1) an Act1-tobacco etch virus (TEV) protease fusion protein, which upon active IL-17A/F binding to the IL-17RA/RC receptor heterodimer on a target cell, will be recruited to (2) IL-17RC fused with a TEV cleavage site(TSC)-tTA (a transcriptional transactivator that binds to tetracycline responsive element, or TRE), releasing tTA, which will subsequently translocate into the nucleus where a (3) TRE-dmCherry-PEST (destabilized mCherry) element is integrated into the genome. tTA binds to the TRE to drive dmCherry expression in the target modified cell. The three described components, Act1-TEV, IL-17RC-TSC-tTA, and TRE-dmCherry-PEST, along with IL-17RA, can be delivered by retro or lenti-viral vectors or guide-RNA directed nucleases, or recombinase systems, or by homologous recombination, into mouse embryonic fibroblasts (MEF) or other mammalian cell lines, establishing stable reporter cell lines. Using the same principle, IL-17RC-TSC-tTA can be replaced with IL-17RB-TSC-tTA in the described system to provide for IL-17E (IL-25) binding, and with IL-17RE-TSC-tTA for detecting IL-17C binding. A parallel approach involves attaching the fusion protein of —TSC-tTA to the RA component and three independent system cell lines generated by supplementing RC/RB/RE, Act1-TEV, and TRE-dmCherry reporter components. Each of the retroviral vectors can contain a unique selection or fluorescent marker to enable efficient generation and maintenance of stable cell lines (for example, Puromycin resistance for m/hIL-17RC(RB, RE)-TSC-tTA; EBFP for m/hAct1-TEV; Neomycin resistance and mVenus fluorescence for TRE-dmCherry-PEST, and Thy1.1 for m/hRA).

The mIL-17A/F system shown in the Figures demonstrated dose-dependent response with a sensitivity of EC50=3.556 ng/ml for IL-17A. It is also a reversible reporter with a half-life of the dmCherry signal of 24 hrs. The described system specifically designed for mIL-17A/F is not responsive to mIL-17E. All three subsystems of the described systems can be robustly used as cross-comparison controls for specific drug screening.

What is claimed is:

1. Modified cells for use in a method for identifying modulators of Interleukin 17 (IL-17) receptors, wherein:
i) the modified cells express an IL-17 receptor that forms a heteromer comprising separate proteins upon activation by ligand binding, wherein at least one of the proteins that forms the heteromer is configured as a first fusion protein that includes a protease recognition site and a transcription transactivator segment, said protease recognition site being configured to liberate the transcription transactivator upon protease cleavage of the protease recognition site;
ii) wherein the cells express an activating protein configured as a second fusion protein, wherein the second fusion protein localizes to the heteromer when formed, and wherein the second fusion protein comprises an activating protein and a protease that is functional on the protease recognition site;
iii) wherein the cells further comprise a sequence encoding a reporter protein that is operably linked to a promoter that is functional with the transcriptional transactivator once liberated from the first fusion protein such that expression of the reporter protein driven by the liberated transcriptional activator signifies activation of the receptor.

2. The modified cells of claim 1, wherein the heteromer comprises a heterodimer.

3. The modified cells of claim 2, wherein the IL-17 receptor can be activated by IL-17A, IL-17F, IL-17E, IL-17C, or a combination thereof.

4. The modified cells of claim 3, wherein the first fusion protein comprises a transcription transactivator that is functional with a tetracycline responsive element.

5. The modified cells of claim 4, wherein the second fusion protein comprises an Act1-tobacco etch virus (TEV) protease that is functional to cleave the first fusion protein to thereby liberate the transcription transactivator.

6. The modified cells of claim 5, wherein the reporter protein participates in production of an optically detectable signal.

7. The modified cells of claim 6, wherein the optically detectable signal is a fluorescent or chemiluminescent signal.

8. A method comprising contacting the modified cells of claim 1 with an agonist of the IL-17 receptor to produce expression of the reporter protein, and subsequently contacting the cells with one or more test agents, wherein a decrease of a signal produced by the reporter protein that is caused by competition between a test agent and the agonist for binding to the IL-17 receptor signifies that the test agent is an antagonist of the IL-17 receptor.

9. The method of claim 8, wherein the heteromer comprises a heterodimer.

10. The method of claim 9, wherein the agonist of the IL-17 is at least one of IL-17A, IL-17F, IL-17E, or IL-17C.

11. The method of claim 10, wherein the first fusion protein comprises a transcription transactivator that is functional with a tetracycline responsive element.

12. The method of claim 11, wherein the second fusion protein comprises an Act1-tobacco etch virus (TEV) protease that is functional to cleave the first fusion protein to thereby liberate the transcription transactivator.

13. The method of claim 12, wherein the reporter protein participates in production of an optically detectable signal.

14. The method of claim 13, wherein the optically detectable signal is a fluorescent or chemiluminescent signal.

15. A method comprising contacting the modified cells of claim 1 with one or more test agents, and wherein detecting expression of the reporter protein signifies that the test agent is an agonist of the receptor.

16. The method of claim 15, wherein the modified cells are present in a plurality of reaction chambers, and wherein optionally each reaction chamber is comprises a different test agent.

17. The method of claim 16, wherein the heteromer comprises a heterodimer.

18. The method of claim 17, wherein the first fusion protein comprises a transcription transactivator that is functional with a tetracycline responsive element.

19. The method of claim 18, wherein the second fusion protein comprises an Act1-tobacco etch virus (TEV) protease that is functional to cleave the first fusion protein to thereby liberate the transcription transactivator.

20. The method of claim 19, wherein the reporter protein participates in production of an optically detectable signal.
